(19) United States
(12) Patent Application Publication
(10) Pub. No.: US 2024/0228428 A1
(43) Pub. Date: Jul. 11, 2024

(54) METHOD FOR PRODUCING ORGANIC SOLVENT SOLUTION OF QUATERNARY AMMONIUM HYDROXIDE

(71) Applicant: TOKUYAMA CORPORATION, Shunan-shi (JP)

(72) Inventors: Shoji TACHIBANA, Shunan-shi (JP); Seiji TONO, Shunan-shi (JP); Sumito ISHIZU, Shunan-shi (JP); Yoshiaki YAMASHITA, Shunan-shi (JP)

(73) Assignee: TOKUYAMA CORPORATION, Shunan-shi (JP)

(21) Appl. No.: 18/615,802

(22) Filed: Mar. 25, 2024

Related U.S. Application Data
(62) Division of application No. 17/280,328, filed on Mar. 26, 2021.

(30) Foreign Application Priority Data
Sep. 28, 2018 (JP) 2018-183274
Jan. 23, 2019 (JP) 2019-009734
Feb. 28, 2019 (JP) 2019-035854

(51) Int. Cl. C07C 211/63

(52) U.S. Cl. CPC C07C 211/63

(57) ABSTRACT

A treatment liquid composition for semiconductor production including: a quaternary ammonium hydroxide; and a first organic solvent dissolving the quaternary ammonium hydroxide, the first organic solvent being a water-soluble organic solvent having a plurality of hydroxy groups, wherein a water content in the composition is no more than 1.0 mass % on the basis of the total mass of the composition; contents of Na, Mg, Al, K, Ca, Ti, Cr, Mn, Fe, Ni, Cu, and Zn in the composition are each no more than 100 mass ppb on the basis of the total mass of the composition; and a content of Cl in the composition is no more than 100 mass ppb on the basis of the total mass of the composition.

This application is a Divisional of co-pending application Ser. No. 17/280,328 filed on Mar. 26, 2021, which is the National Stage Application under 35 U.S.C. § 371 of International Application No. PCT/JP2019/038008, filed on Sep. 26, 2019, which claims the benefit under 35 U.S.C. § 119(a) to Japanese Patent Application No. 2018-183274, filed on Sep. 28, 2018, Japanese Patent Application No. 2019-035854, filed on Feb. 28, 2019, and Japanese Patent Application No. 2019-009734, filed on Jan. 23, 2019, all of which are hereby expressly incorporated by reference into the present application.

### FIELD

The present invention relates to a method for producing an organic solvent solution of a quaternary ammonium hydroxide, and to a treatment liquid composition for semiconductor production and a method for producing the same.

### BACKGROUND

Solutions containing a quaternary ammonium hydroxide are used as developers for photoresists (may be simply referred to as “resists”), strippers and cleaning solutions for modified photoresists (such as photoresists after an ion implantation process, and photoresists after ashing), silicon etchants, etc. in production processes of a semiconductor devices, liquid crystal displays, etc.

For example, in the development process of a photoresist, a negative or positive photoresist containing a resin such as novolac resins and polystyrene resins is applied to the surface of a substrate. The applied photoresist is irradiated with light via a photomask for pattern generation, which cures or solubilizes the irradiated photoresist. Part of the photoresist which does not cure or which is solubilized is removed using a developer, to form a photoresist pattern.

The formed photoresist pattern plays a role so that any portion that is not covered with the photoresist pattern is selectively treated in the subsequent process (such as etching, doping, and ion implantation). Thereafter the photoresist pattern, which will not be used anymore, is removed from the surface of the substrate by a resist stripper after ashed as necessary. The substrate is further cleaned with a cleaning solution so as to remove residue of the resist if necessary.

Aqueous quaternary ammonium hydroxide solutions are conventionally used for these purposes. A photoresist pattern changes properties thereof after being subjected to a process such as ion implantation, which leads to formation of a carbonaceous crust on the surface thereof. A modified photoresist where the crust is formed on the surface thereof is not easy to remove by a conventional aqueous quaternary ammonium hydroxide solution. Ashed residue of a photoresist pattern also has properties similar to carbonaceous matters, and is not easy to remove by a conventional aqueous quaternary ammonium hydroxide solution.

For the purpose of more effectively removing such a modified photoresist or residue of an ashed photoresist, it is proposed to use an organic solvent solution of a quaternary ammonium hydroxide instead of an aqueous quaternary ammonium hydroxide solution. An organic solvent solution of a quaternary ammonium hydroxide is also advantageous in that the organic solvent solution seldom corrodes metallic materials used for wiring, or inorganic substrate materials such as Si, SiOₓ, SiNₓ, Al, TiN, W and Ta, compared to an aqueous quaternary ammonium hydroxide solution.

### CITATION LIST

### Patent Literature

Patent Literature 1: JP 4673935 B2
Patent Literature 2: JP 4224651 B2
Patent Literature 3: JP 4678673 B2
Patent Literature 4: JP 6165442 B2
Patent Literature 5: WO 2016/163384 A1
Patent Literature 6: WO 2017/169832 A1

### SUMMARY

### Technical Problem

The water content of an organic solvent solution of a quaternary ammonium hydroxide is desirably low in view of enhancement of the removing performance for a modified photoresist or residue of an ashed photoresist, and the compatibility with metallic materials and inorganic substrate materials. The impurity metal content of an organic solvent solution of a quaternary ammonium hydroxide is desirably low as well in view of improving yields of semiconductor devices.

For example, tetramethylammonium hydroxide (TMAH), which is one of quaternary ammonium hydroxides, is commercially available as a 2.38 to 25 mass % aqueous solution, or as a crystalline solid of TMAH pentahydrate (approximately 97 to 98 mass % in purity). However, anhydrous TMAH that substantially contains no water is not commercially distributed.

Generally, quaternary ammonium hydroxides are produced by electrolyzing an aqueous solution of a quaternary ammonium halide such as tetramethylammonium chloride (TMAC) (electrolysis method). This electrolysis results in exchange of a halide ion, which is a counter ion of a quaternary ammonium ion, for a hydroxide ion, to produce an aqueous quaternary ammonium hydroxide solution. For example, the concentration of a TMAH aqueous solution produced by the electrolysis method is usually approximately 20 to 25 mass %. The electrolysis method offers production of an aqueous quaternary ammonium hydroxide solution of a high degree of purity which contains metal impurities in an amount of approximately no more than 0.1 mass ppm in terms of each metal, and in particular, offers production of an aqueous TMAH solution of a high degree of purity which contains metal impurities in an amount of approximately no more than 0.001 mass ppm (that is, no more than 1 mass ppb) in terms of each metal.

Unfortunately, it is extremely difficult to obtain an anhydrous quaternary ammonium hydroxide from an aqueous quaternary ammonium hydroxide solution. For example, a higher concentration of a TMAH aqueous solution leads to precipitation of the crystalline solid of TMAH pentahydrate (TMAH content: approximately 50 mass %). Even if the crystalline solid of TMAH pentahydrate is heated, TMAH trihydrate (TMAH content: approximately 63 mass %) may be formed on one hand, but at the same time decomposition of TMAH (formation and liberation of trimethylamine) proceeds on the other hand.

The counterion exchange method is known as a method for producing an organic solvent solution of a quaternary ammonium hydroxide. For example, tetramethylammonium chloride (TMAC) and potassium hydroxide (KOH) are mixed with each other in methanol, to form TMAH and potassium chloride (KCl), and KCl precipitates due to its low solubility in methanol. The KCl precipitate is filtered off, to give a TMAH methanolic solution. The counterion exchange method offers production of a TMAH methanolic solution that has a relatively low water content on one hand, but this solution contains 0.5 to several mass % of impurities such as KCl and water on the other hand. Thus, the counterion exchange method cannot give a TMAH methanolic solution of a high degree of purity which is useful in the production process of semiconductors.

As another method for producing an organic solvent solution of a quaternary ammonium hydroxide, Patent Literature 1 describes a process for producing a concentrate of a quaternary ammonium hydroxide comprising: mixing a quaternary ammonium hydroxide in a form of a hydrate crystal or an aqueous solution with a water-soluble organic solvent selected from the group consisting of glycol ethers, glycols, and triols, to prepare a mixed solution; and subjecting this mixed solution to a thin-film evaporation under reduced pressure, to evaporate a low-boiling material off. Patent Literature 1 asserts that, for example, a propylene glycol solution of TMAH (TMAH content: 12.6 mass %, water content: 2.0 mass %) was obtained by thin-film distillation using a 25 mass % TMAH aqueous solution as a starting material.

Unfortunately, in a supplementary examination of the method described in Patent Literature 1 conducted by the present inventors using an aqueous quaternary ammonium hydroxide solution of a high degree of purity as a starting material, metal impurities in an amount much more than 0.1 mass ppm were detected from an organic solvent solution of the quaternary ammonium hydroxide obtained by thin film evaporation. The impurity metal content in an organic solvent solution of a quaternary ammonium hydroxide is desirably, at most, no more than 0.1 mass ppm in terms of each metal in view of the use in the production process of semiconductor devices.

As described above, an organic solvent solution of a quaternary ammonium hydroxide of a sufficiently high degree of purity in view of the use in the production process of semiconductors has not been obtained yet.

An object of the present invention is to provide a treatment liquid composition for semiconductor production which is based on an organic solvent solution of a quaternary ammonium hydroxide of such a high degree of purity that the composition is useful for the production processes of semiconductors. A method for producing an organic solvent solution of a quaternary ammonium hydroxide, and a method for producing a treatment liquid composition for semiconductor production are also provided.

### Solution to Problem

The present invention encompasses the following [1] to [17].
[1] A treatment liquid composition for semiconductor production, the composition comprising:
a quaternary ammonium hydroxide; and
a first organic solvent dissolving the quaternary ammonium hydroxide, the first organic solvent being a water-soluble organic solvent having a plurality of hydroxy groups,
wherein a water content in the composition is no more than 1.0 mass % on the basis of the total mass of the composition;
contents of Na, Mg, Al, K, Ca, Ti, Cr, Mn, Fe, Ni, Cu, and Zn in the composition are each no more than 100 mass ppb on the basis of the total mass of the composition; and
a content of Cl in the composition is no more than 100 mass ppb on the basis of the total mass of the composition.
[2] The composition according to [1],
wherein the water content in the composition is no more than 0.5 mass % on the basis of the total mass of the composition;
the contents of Na, Mg, Al, K, Ca, Ti, Cr, Mn, Fe, Ni, Cu, and Zn in the composition are each no more than 50 mass ppb on the basis of the total mass of the composition; and
the content of Cl in the composition is no more than 80 mass ppb on the basis of the total mass of the composition.
[3] The composition according to [1] or [2],
wherein the water content in the composition is no more than 0.3 mass % on the basis of the total mass of the composition;
the contents of Na, Mg, Al, K, Ca, Ti, Cr, Mn, Fe, Ni, Cu, and Zn in the composition are each no more than 20 mass ppb on the basis of the total mass of the composition; and
the content of Cl in the composition is no more than 50 mass ppb on the basis of the total mass of the composition.
[4] The composition according to any one of [1] to [3],
wherein a content of the quaternary ammonium hydroxide in the composition is no less than 5.0 mass % on the basis of the total mass of the composition.
[5] The composition according to any one of [1] to [3],
wherein a content of the quaternary ammonium hydroxide in the composition is 2.38 to 25.0 mass % on the basis of the total mass of the composition; and
the quaternary ammonium hydroxide is tetramethylammonium hydroxide.
[6] The composition according to any one of [1] to [5],
wherein the first organic solvent is at least one alcohol selected from divalent alcohols and trivalent alcohols, wherein each of the divalent alcohols and trivalent alcohols consists of carbon atoms, hydrogen atoms, and oxygen atoms, and wherein each of the divalent alcohols and trivalent alcohols has a boiling point of 150 to 300° C.
[7] A method for producing an organic solvent solution of a quaternary ammonium hydroxide,
the solution having a water content of no more than 1.0 mass % on the basis of the total mass of the solution,
contents of Na, Mg, Al, K, Ca, Ti, Cr, Mn, Fe, Ni, Cu, and Zn in the solution each being no more than 100 mass ppb on the basis of the total mass of the solution,
the solution having a Cl content of no more than 100 mass ppb on the basis of the total mass of the solution,
the method comprising:
   (a) subjecting a raw material mixture liquid to a thin film evaporation by means of a thin film evaporation apparatus, to remove water from the raw material mixture liquid,
the raw material mixture liquid comprising:
   a quaternary ammonium hydroxide;
   water; and
   a first organic solvent dissolving the quaternary ammonium hydroxide, the first organic solvent being a water-soluble organic solvent having a plurality of hydroxy groups,
the thin film evaporation apparatus comprising:
   an evaporation vessel;
   a raw material reservoir storing the raw material mixture liquid; and
   a raw material conduit transferring the raw material mixture liquid from the raw material reservoir to the evaporation vessel,
wherein liquid-contacting portions of inner surfaces of the raw material reservoir and the raw material conduit are each made of resin.
[8] The method according to [7],
wherein contents of Na, Ca, Al, and Fe in the resin constituting the liquid-contacting portions are each no more than 1 mass ppm.
[9] The method according to [7] or [8], the method further comprising:
(b) prior to the (a), washing the liquid-contacting portions with a solution comprising the quaternary ammonium hydroxide.
[10] The method according to any one of [7] to [9],
wherein the first organic solvent has a boiling point of 150 to 300° C.
[11] The method according to any one of [7] to [10],
wherein the first organic solvent is at least one alcohol selected from divalent alcohols and trivalent alcohols, wherein each of the divalent alcohols and trivalent alcohols consists of carbon atoms, hydrogen atoms, and oxygen atoms, and wherein each of the divalent alcohols and trivalent alcohols has a boiling point of 150 to 300° C.
[12] The method according to any one of [7] to [11],
wherein the first organic solvent is ethylene glycol, propylene glycol, diethylene glycol, dipropylene glycol, tripropylene glycol, hexylene glycol, or glycerin, or any combination thereof.
[13] The method according to any one of [7] to [12],
the raw material mixture liquid comprising, on the basis of the total mass of the mixture liquid:
   40 to 85 mass % of the first organic solvent;
   2.0 to 30 mass % of the quaternary ammonium hydroxide; and
   10 to 30 mass % of the water.
[14] The method according to any one of [7] to [13],
wherein contents of Na, Mg, Al, K, Ca, Ti, Cr, Mn, Fe, Ni, Cu, and Zn in the raw material mixture liquid are each no more than 50 mass ppb on the basis of the total mass of the raw material mixture liquid; and
a content of Cl in the raw material mixture liquid is no more than 50 mass ppb on the basis of the total mass of the raw material mixture liquid.
[15] The method according to any one of [7] to [14],
the thin film evaporation apparatus being a flowing-down-type thin film evaporation apparatus,
the evaporation vessel comprising an inner wall surface,
the thin film evaporation apparatus further comprising:
   a first flow path introducing the raw material mixture liquid into the evaporation vessel from an upper part of the evaporation vessel,
the raw material mixture liquid introduced from the first flow path into the evaporation vessel flowing down as a liquid film along the inner wall surface of the evaporation vessel,
the thin film evaporation apparatus further comprising:
   a heating surface arranged in the inner wall surface, the heating surface heating the liquid film flowing down along the inner wall surface;
   a condenser arranged inside evaporation vessel, the condenser cooling and liquefying a vapor from the liquid film;
   a second flow path recovering a distillate liquefied by the condenser from the evaporation vessel; and
   a third flow path recovering a residue from the evaporation vessel, the residue not evaporating but flowing down from the heating surface,
wherein the thin film evaporation is carried out under conditions such that:
   the raw material mixture liquid has a first temperature right before entering into the evaporation vessel, wherein the first temperature is no more than 70° C.;
   the heating surface has a second temperature of 60 to 140° C., wherein the second temperature is higher than the first temperature; and
   a degree of vacuum in the evaporation vessel is no more than 600 Pa.
[16] The method according to [15],
the thin film evaporation apparatus further comprising:
   a wiper being arranged in the evaporation vessel and rotating along the inner wall surface,
wherein the raw material mixture liquid introduced into the evaporation vessel from the first flow path is spread on the inner wall surface with the wiper, to form the liquid film.
[17] A method for producing a treatment liquid composition for semiconductor production, the method comprising:
(i) obtaining an organic solvent solution of a quaternary ammonium hydroxide by a method as in any one of [7] to [16];
(ii) knowing a concentration of the quaternary ammonium hydroxide in the organic solvent solution; and
(iii) adding a second organic solvent to the organic solvent solution, to adjust the concentration of the quaternary ammonium hydroxide in the organic solvent solution, wherein the second organic solvent has a water content of no more than 1.0 mass % on the basis of the total mass of the second organic solvent, and wherein contents of Na, Mg, Al, K, Ca, Ti, Cr, Mn, Fe, Ni, Cu, and Zn in the second organic solvent are each no more than 100 mass ppb on the basis of the total mass of the second organic solvent, and wherein the second organic solvent has a Cl content of no more than 100 mass ppb on the basis of the total mass of the second organic solvent,
wherein the composition is a treatment liquid composition for semiconductor production as in any one of [1] to [6].

### Advantageous Effects of Invention

The treatment liquid composition for semiconductor production according to the first aspect of the present invention makes it possible to provide a treatment liquid composition for semiconductor production which is based on an organic solvent solution of a quaternary ammonium hydroxide of such a high degree of purity that the composition is useful for the production processes of semiconductors.

The method for producing an organic solvent solution of a quaternary ammonium hydroxide according to the second aspect of the present invention makes it possible to produce an organic solvent solution of a quaternary ammonium hydroxide of a high degree of purity which may be preferably used as the treatment liquid composition for semiconductor production according to the first aspect of the present invention, or which may be preferably used for production of the treatment liquid composition for semiconductor production according to the first aspect of the present invention.

The method for producing a treatment liquid composition for semiconductor production according to the third aspect of the present invention makes it possible to preferably produce the treatment liquid composition for semiconductor production according to the first aspect of the present invention.

### BRIEF DESCRIPTION OF DRAWINGS

FIG. 1 is an explanatorily schematic view of a flowing-down-type thin film evaporation apparatus 10A according to one embodiment.

FIG. 2 is a schematically explanatory cross-sectional view of an evaporation vessel 37 in the apparatus 10A in detail.

FIG. 3 is an explanatorily schematic view of a thin film evaporation apparatus 10B according to another embodiment.

FIG. 4 is an explanatorily schematic view of a thin film evaporation apparatus 10C according to still another embodiment.

### DETAILED DESCRIPTION OF EMBODIMENTS

The foregoing effects and advantages of the present invention will be made clear from the following description of the embodiments. Hereinafter the embodiments of the present invention will be described with reference to the drawings. The present invention is not limited to these embodiments. The measures in the drawings do not always represent the exact measures. Some reference signs and hatching may be omitted in the drawings. In the present description, expression “A to B” concerning numeral values A and B shall mean “no less than A and no more than B” unless otherwise specified. In such expression, if a unit is added only to the numeral value B, the same unit shall be applied to the numeral value A as well. A word “or” shall mean a logical sum unless otherwise specified. Expression “E₁ and/or E₂” concerning elements E₁ and E₂ means “E₁, or E₂, or the combination thereof”, and expression “E₁, . . . , E_{N-1}, and/or E_{N}” concerning elements E₁, . . . , E_{N} (N is an integer of 3 or more) means “E₁, . . . , E_{N-1}, or E_{N}, or any combination thereof”.

### <1. Treatment Liquid Composition for Semiconductor Production>

A treatment liquid composition for semiconductor production according to the first aspect of the present invention (hereinafter may be simply referred to as “composition”) comprises a quaternary ammonium hydroxide, and a first organic solvent dissolving the quaternary ammonium hydroxide. The first organic solvent is a water-soluble organic solvent having a plurality of hydroxy groups.

### (1.1 Quaternary Ammonium Hydroxide)

A quaternary ammonium hydroxide (hereinafter may be referred to as “QAH”) is an ionic compound constituted of an ammonium cation and a hydroxide ion (anion). The ammonium cation comprises a nitrogen atom and four organic groups bonded to the nitrogen atom. The composition according to the present invention may comprise only one quaternary ammonium hydroxide, or may comprise two or more quaternary ammonium hydroxides. Examples of a quaternary ammonium hydroxide include compounds represented by the following general formula (1)

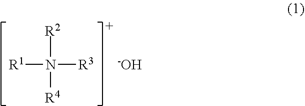

In general formula (1), R¹ to R⁴ are each independently a hydrocarbon group that may have a hydroxy group, preferably an alkyl group that may have a hydroxy group. In view of, the removing performance for resists and modified resists, and the etching performance, etc., R¹ to R⁴ are especially preferably C₁₋₄ alkyl groups that may have a hydroxy group. Specific examples of R¹ to R⁴ include a methyl group, an ethyl group, a propyl group, a butyl group, and a 2-hydroxyethyl group.

In the general formula (1), R¹ to R⁴ may be the same, or may be different from one another. In one embodiment, R¹ to R⁴ may be the same group, preferably a C₁₋₄ alkyl group. In another embodiment, R¹ to R³ may be the same group (first group) and R⁴ may be a group (second group) different from R¹ to R³. In one embodiment, the first and second groups may be each independently a C₁₋₄ alkyl group. In another embodiment, the first group may be a C₁₋₄ alkyl group and the second group may be a C₁₋₄ hydroxyalkyl group.

Specific examples of a quaternary ammonium hydroxide include tetramethylammonium hydroxide (TMAH), tetraethylammonium hydroxide (TEAH), tetrapropylammonium hydroxide (TPAH), tetrabutylammonium hydroxide (TBAH), and trimethyl-2-hydroxyethylammonium hydroxide (synonym: choline hydroxide).

Among them, TMAH is especially preferable because it is particularly excellent in the removing performance for resists and modified resists, and the etching performance, etc., and is inexpensive and versatile. Any compound obtained by substituting a part or all of the methyl groups in TMAH for (an)other group(s) such as an ethyl group, a propyl group and a butyl group, i.e., TEAH, TPAH, TBAH, choline hydroxide as described above may be preferred at the production site for semiconductor devices in view of not being toxic and the compatibility with resist materials to be used, although they are inferior to TMAH in the removing performance for resists and modified resists, and the etching performance, etc.

In one embodiment, the content of the quaternary ammonium hydroxide in the composition may be 2.38 to 25.0 mass %. In one preferred embodiment, TMAH may be used as the quaternary ammonium hydroxide. The TMAH content in the composition may be 2.38 to 25.0 mass % on the basis of the total mass of the composition.

In one embodiment, the content of the quaternary ammonium hydroxide in the composition may be preferably no less than 5.0 mass %, and more preferably no less than 8.0 mass %, on the basis of the total mass of the composition. The content of the quaternary ammonium hydroxide in the composition at the above lower limit or more makes it possible to save the distribution cost for the composition. The upper limit of this content is not particularly limited, but may be no more than 72 mass % in one embodiment, and no more than 55 mass % in another embodiment. The content of the quaternary ammonium hydroxide in the composition at the above upper limit or less offers suppression of viscosity increase of the composition, which makes it easy to, e.g., handle, feed, and mix the composition when the composition is used.

The concentration of the quaternary ammonium hydroxide in the composition can be accurately measured with a potentiometric titration apparatus, liquid chromatography, etc. These measuring means may be used alone, or may be used in combination.

### (1.2 First Organic Solvent)

The composition according to the present invention comprises, as a solvent, the first organic solvent dissolving the quaternary ammonium hydroxide. The first organic solvent is a water-soluble organic solvent having a plurality of hydroxy groups. As the first organic solvent, one solvent may be used alone, or two or more solvents may be used in combination.

The water content in the composition can be reduced by evaporating water from the composition, since a water-soluble organic solvent having two or more hydroxy groups has a higher boiling point than water. The boiling point of the first organic solvent at 0. 1 MPa in pressure is preferably 150 to 300° C., and more preferably 150 to 200° C. The boiling point of the first organic solvent no less than 150° C. makes it difficult for the first organic solvent to evaporate when water is evaporated off, which makes it easy to reduce the water content in the composition. The first organic solvent having a boiling point at the above upper limit or lower does not have so high viscosity, which makes it possible to increase efficiency when water is evaporated off.

As the first organic solvent, at least one alcohol selected from divalent alcohols and trivalent alcohols, more preferably divalent aliphatic alcohols and trivalent aliphatic alcohols, each consisting of carbon atoms, hydrogen atoms, and oxygen atoms, and each having a boiling point of 150 to 300° C. may be preferably used. The melting point of the first organic solvent is preferably no more than 25° C., and more preferably no more than 20° C.

Specific examples of the preferred first organic solvents include divalent alcohols such as ethylene glycol (boiling point: 197° C.), propylene glycol (boiling point: 188° C.), diethylene glycol (boiling point: 244° C.), dipropylene glycol (boiling point: 232° C.), tripropylene glycol (boiling point: 267° C.) and hexylene glycol (2-methyl-2,4-pentanediol) (boiling point: 198° C.); and trivalent alcohols such as glycerin (boiling point: 290° C.); and any combinations thereof.

Among them, an alcohol having a hydroxy group bonding to a secondary or tertiary carbon atom such as propylene glycol, dipropylene glycol, tripropylene glycol and hexylene glycol may be preferably used as the first organic solvent in view of the storage stability of the composition. Among them, propylene glycol and hexylene glycol are especially preferable in view of the foregoing boiling point and the storage stability of the composition, and in view of availability and cost as well.

### (1.3 Second Organic Solvent)

The composition according to the present invention may further comprise an organic solvent (hereinafter may be referred to as “second organic solvent”) other than the water-soluble organic solvent having a plurality of hydroxy groups, according to what is to be treated with the composition. Examples of the second organic solvent include organic solvents known as organic solvents incorporated in a treatment liquid composition for semiconductor production. Preferred examples of the second organic solvent include water-soluble organic solvents each having only one hydroxy group (water-soluble monovalent alcohols) such as methanol, ethanol, 1-propanol, 2-propanol and n-butanol. These water-soluble monovalent alcohols each having only one hydroxy group may be preferably used for, for example, adjusting the viscosity of the composition.

The proportion of the first organic solvent to the total organic solvent in the composition according to the present invention is preferably no less than 50 mass %, more preferably no less than 75 mass %, further preferably no less than 95 mass %, and especially preferably 100 mass % substantially, on the basis of the total mass of the organic solvent. Here, the proportion of the first organic solvent to the total organic solvent in the composition being “100 mass % substantially” means that the total organic solvent in the composition is constituted of the first organic solvent only, or that the total organic solvent in the composition is constituted of the first organic solvent and inevitable impurities.

### (1.4 Water Content in Composition)

The water content in the composition is no more than 1.0 mass %, preferably no more than 0.5 mass %, and more preferably no more than 0.3 mass %, on the basis of the total mass of the composition. The water content in the composition at the above upper limit or less can enhance the removing performance for modified photoresists and residue of ashed photoresists, and can reduce the corrosivity to metallic materials and inorganic substrate materials. The lower limit of the water content in the composition is not particularly limited, but may be, for example, no less than 0.05 mass %.

The water content in the composition can be measured by gas chromatography, or with a Karl Fischer titrator using the Karl Fischer method (hereinafter may be referred to as “Karl Fischer titration”) and gas chromatography in combination. A Karl Fischer titrator makes measurement in a simple operation possible. However, measurements by Karl Fischer titration may contain an error due to an interfering reaction in the presence of an alkali. In contrast, gas chromatography makes it possible to accurately measure the water content regardless of the presence or absence of an alkali, but the measurement operation thereof is not exactly simple. As for a solution having the alkali concentration approximately same as that of the composition, water content measurements from a Karl Fischer titrator are plotted on the vertical axis, and water content measurements from gas chromatography are plotted on the horizontal axis, to draw a calibration curve in advance; and the measurements from a Karl Fischer titrator are corrected using this calibration curve, which makes it possible to accurately quantify the water content in a simple operation. Commercially available apparatuses may be used for gas chromatography and as a Karl Fischer titrator.

The operation of correcting the water content measurements from a Karl Fischer titrator using a calibration curve may be preferably carried out through the following procedures (1) to (6).
(1) The water content in an organic solvent that is the same as the organic solvent in the composition to be measured is measured by Karl Fischer titration. Water is added to this organic solvent to prepare, e.g., five solutions of different water contents (hereinafter may be referred to as “water/organic solvent solutions”). The amount of water added to the organic solvent is selected so that the water content in the composition to be measured is within the range of the water contents in the water/organic solvent solutions. For example, when the water content in the composition to be measured is considered to be 0.05 to 5.0 mass %, the amount of water added to the organic solvent can be determined so that the water contents in the water/organic solvent solutions are five levels of 0.05 to 5.0 mass %. It is desirable to measure the water contents in the five prepared water/organic solvent solutions by Karl Fischer titration, and confirm that the obtained values well match the theoretical values calculated from the water content in the organic solvent and the amount of the added water.
(2) Each of the five water/organic solvent solutions prepared in (1) is analyzed by gas chromatography (hereinafter may be referred to as “GC”), so that GC charts including the peaks of water and the organic solvents are obtained. The areas of the peaks of water in the obtained GC charts are plotted on the vertical axis (Y), and the water contents in the water/organic solvent solutions (theoretical values calculated from the water content in the organic solvent and the amount of the added water) are plotted on the horizontal axis (X). A regression line is calculated by the least squares using Y as a dependent variable and X as an explanatory variable, so that a calibration curve that gives the water content from the areas of the peaks of water in the GC charts (hereinafter may be referred to as “first calibration curve”) is obtained.
(3) A concentrated aqueous solution of QAH, which is the same as the quaternary ammonium hydroxide (QAH) in the composition to be measured (the QAH concentration in the concentrated aqueous solution has only to be as high as available, and may be, for example, 10 to 25 mass %), is added to the organic solvent same as the organic solvent in the composition to be measured, so that five mixed solutions are prepared as standard solutions. The water content in the organic solvent is accurately measured by Karl Fischer titration in (1). The QAH concentration in the concentrated aqueous solution of QAH is accurately measured with an automatic potentiometric titration apparatus. This also determines the water content in the concentrated aqueous solution of QAH at the same time. The mixing mass ratio of the organic solvent and the concentrated aqueous solution of QAH is selected so that the water contents in the mixed solutions are five levels that are the same as in (1).
(4) Each of the five standard solutions prepared in (3) is analyzed by gas chromatography, so that the water content in each of the standard solutions is obtained from the areas of the peaks of water in the GC charts, using the first calibration curve obtained in (2). Generally, a water content measurement from GC well matches a theoretical value of the water content in a standard solution which is calculated from the water content in an organic solvent, the water content in a concentrated aqueous solution of QAH, and the mixing mass ratio of the organic solvent and the concentrated aqueous solution of QAH.
(5) The water content in each of the five standard solutions prepared in (3) is measured by Karl Fischer titration. The water contents in the standard solutions measured by Karl Fischer titration are plotted on the vertical axis (Y), and the water contents in the standard solutions, which are measured by GC in (3), are plotted on the horizontal axis (X). A regression line is calculated by the least squares using Y as a dependent variable and X as an explanatory variable, so that a calibration curve for correcting a water content measurement of the organic solvent solution containing QAH and water from Karl Fischer titration to that from GC (hereinafter may be referred to as “second calibration curve”) is obtained.
(6) The water content of the actual composition to be measured is measured by Karl Fischer titration. The obtained measurement is corrected to the water content measured by GC, using the second calibration curve obtained in (5).

It is not essential to measure the water content in the composition by Karl Fischer titration. Using the first calibration curve obtained by the above procedures (1) to (2) makes it possible to accurately measure the water content in the composition containing an alkali by gas chromatography analysis.

The ratio of the water content in the composition (unit: mass %) to the content of the quaternary ammonium hydroxide in the composition (unit: mass %) (water content/content of the quaternary ammonium hydroxide) is preferably no more than 0.42, more preferably no more than 0.21, and further preferably no more than 0.10. This ratio at the above upper limit or less makes it possible to maintain or improve the removing performance for modified photoresists and residue of ashed photoresists, and at the same time to further reduce the corrosivity to metallic materials and inorganic substrate materials. The lower limit of this ratio is not particularly limited, but may be, for example, no less than 0.0007.

### (1.5 Impurities in Composition)

The metal impurity content in the composition is no more than 100 mass ppb, preferably no more than 50 mass ppb, and more preferably no more than 20 mass ppb, in terms of Na, Mg, Al, K, Ca, Ti, Cr, Mn, Fe, Ni, Cu and Zn each on the basis of the total mass of the composition. In the present description, the metal impurity content in the composition means the total content of metal elements in the metal impurities regardless of whether each of the metal elements is a zero-valent metal or in the form of a metal ion.

The chlorine impurity (Cl) content in the composition is no more than 100 mass ppb, preferably no more than 80 mass ppb, and more preferably no more than 50 mass ppb, on the basis of the total mass of the composition. In the present description, the chlorine impurity content in the composition means the total content of a chlorine element. In the composition, chlorine impurities are usually present in the form of a chloride ion (Cl⁻).

The metal impurity content in the composition can be measured with a microanalyzer such as an inductively coupled plasma mass spectrometer (ICP-MS). The chlorine impurity content can be measured by a microanalyzer using ion chromatography etc.

The ratio of the metal impurity content in the composition (unit: mass ppb) to the content of the quaternary ammonium hydroxide in the composition (unit: mass %) (metal impurity content/content of the quaternary ammonium hydroxide) is preferably no more than 42, more preferably no more than 21, and further preferably no more than 10, in terms of each of the foregoing metal elements. This ratio at the above upper limit or less makes it possible to maintain or improve the removing performance for modified photoresists and residue of ashed photoresists, and at the same time to further increase yields of semiconductor devices. The lower limit of this ratio is not particularly limited, but the lower the more preferable. The lower limit may be, for example, no less than 0.0001 in view of, for example, the quantitative limit of a measurement device for metal impurities. The ratio of the chlorine impurity content in the composition (unit: mass ppb) to the content of the quaternary ammonium hydroxide in the composition (unit: mass %) (chlorine content/content of the quaternary ammonium hydroxide) is preferably no more than 42, more preferably no more than 34, and further preferably no more than 21. This ratio at the above upper limit or less makes it possible to maintain or improve the removing performance for modified photoresists and residue of ashed photoresists, and at the same time to further increase yields of semiconductor devices. The lower limit of this ratio is not particularly limited, but the lower the more preferable. The lower limit may be, for example, no less than 0.001 in view of, for example, the quantitative limit of a measurement device for chlorine impurities.

### (1.6 Use)

For example, the composition according to the present invention may be preferably used as a chemical liquid used in the production process of semiconductor devices, such as developers for photoresists, strippers and cleaning solutions for modified photoresists, and silicon etchants.

In the semiconductor production field, not only the foregoing various chemical liquids themselves but also concentrated liquids that are to be diluted with a solvent or the like to be used for preparing the foregoing various chemical liquids are also referred to as treatment liquids. In the present description, not only compositions having such a concentration as to be capable of being used as they are as the foregoing various chemical liquids but also concentrated liquids to be diluted as described above shall also fall under “treatment liquid composition for semiconductor production”. The composition according to the present invention may be preferably used as the foregoing concentrated liquid as well. For example, the composition according to the present invention is diluted with (the concentration thereof is adjusted by) the first organic solvent, the second organic solvent, water, or an aqueous quaternary ammonium hydroxide solution, or any combination thereof, which makes it possible to obtain a chemical liquid having a desired concentration of the quaternary ammonium hydroxide and a desired solvent composition.

### <2. Method for Producing Organic Solvent Solution of Quaternary Ammonium Hydroxide>

A method for producing an organic solvent solution of a quaternary ammonium hydroxide according to the second aspect of the present invention (hereinafter may be referred to as “solution production method”) comprises a step (a) of subjecting a raw material mixture liquid to a thin film evaporation by means of a thin film evaporation apparatus, to remove water from the raw material mixture liquid (hereinafter, may be referred to as “step (a)”).

### (2.1 Raw Material Mixture Liquid)

The raw material mixture liquid comprises a quaternary ammonium hydroxide (hereinafter may be referred to as “QAH”), water, and a first organic solvent dissolving the quaternary ammonium hydroxide. The first organic solvent is a water-soluble organic solvent having a plurality of hydroxy groups.

### (2.1.1 Quaternary Ammonium Hydroxide)

The quaternary ammonium hydroxide described in the section 1.1 in connection with the composition according to the first aspect of the present invention may be employed as a quaternary ammonium hydroxide in the raw material mixture liquid. A preferred aspect of this quaternary ammonium hydroxide is also the same as in the section 1.1.

### (2.1.2 First Organic Solvent)

The water-soluble organic solvent having a plurality of hydroxy groups described in the section 1.2 in connection with the composition according to the first aspect of the present invention may be employed as the first organic solvent in the raw material mixture liquid. A preferred aspect of this first organic solvent is also the same as in the section 1.2. As the first organic solvent in the raw material mixture liquid, one solvent may be used alone, or two or more solvents may be used in combination.

### (2.1.3 Composition of Raw Material Mixture Liquid)

The proportion of the foregoing three constituents in the raw material mixture liquid is not particularly limited, but the proportion of water is desirably as small as possible. Quaternary ammonium hydroxides that are commercially available currently on an industrial scale are usually manufactured by the electrolysis method, and are often distributed in the form of an aqueous solution. For example, the TMAH concentration of a concentrated aqueous solution of TMAH which is commercially available currently is typically approximately 20 to 25 mass %. For example, the concentrations of concentrated aqueous solutions of TEAH, TPAH, TBAH, and choline hydroxide which are commercially available currently are each typically approximately 10 to 55 mass %. For example, an aqueous quaternary ammonium hydroxide solution and the foregoing water-soluble organic solvent are mixed, so that the raw material mixture liquid can be prepared. The mixing ratio of the quaternary ammonium hydroxide and water in the raw material mixture liquid, which is prepared as described above, reflects the concentration of the used aqueous quaternary ammonium hydroxide solution. In view of reduction of the amount of water to be distilled in the thin film evaporation, the concentration of the aqueous quaternary ammonium hydroxide solution used for preparing the raw material mixture liquid is desirably high. For example, a crystalline solid such as TMAH pentahydrate may be dissolved in the water-soluble organic solvent and used. However, a highly concentrated aqueous quaternary ammonium hydroxide solution and the crystalline solid are often expensive. The water content in the raw material mixture liquid may be determined in view of the cost for obtaining the aqueous quaternary ammonium hydroxide solution or the crystalline solid, the impurity content, etc.

The content of the first organic solvent in the raw material mixture liquid may be, for example, preferably 30 to 85 mass %, more preferably 40 to 85 mass %, further preferably 40 to 80 mass %, and especially preferably 60 to 80 mass %, on the basis of the total mass of the raw material mixture liquid.

The content of the quaternary ammonium hydroxide in the raw material mixture liquid may be, for example, preferably 2.0 to 40 mass %, more preferably 2.0 to 30 mass %, further preferably 2.0 to 25 mass %, and especially preferably 5.0 to 10 mass %, on the basis of the total mass of the raw material mixture liquid. The water content in the raw material mixture liquid may be, for example, preferably 10 to 30 mass %, and more preferably 15 to 30 mass %, on the basis of the total mass of the raw material mixture liquid.

The impurity content in the raw material mixture liquid is desirably low. Particularly, the contents of metal impurities, and nonvolatile impurities such as a chloride ion, a carbonate ion, a nitrate ion, and a sulfate ion are desirably low since such impurities are difficult to remove by the thin film evaporation.

Metal impurities are present as ions or fine particles in a solution. In the present description, metal impurities encompass both metal ions and metal particles. In view of obtainment of the foregoing composition of a high degree of purity, the metal impurity content in the raw material mixture liquid may be, for example, preferably no more than 50 mass ppb, more preferably no more than 20 mass ppb, and further preferably no more than 10 mass ppb, in terms of Na, Mg, Al, K, Ca, Ti, Cr, Mn, Fe, Ni, Cu and Zn each on the basis of the total mass of the raw material mixture liquid.

The chlorine impurity content in the raw material mixture liquid may be, for example, preferably no more than 50 mass ppb, more preferably no more than 30 mass ppb, and further preferably no more than 20 mass ppb, on the basis of the total mass of the raw material mixture liquid.

The metal impurity content in the aqueous quaternary ammonium hydroxide solution used for preparing the raw material mixture liquid is preferably no more than 100 mass ppb, and more preferably no more than 1 mass ppb, in terms of each metal on the basis of the total mass of the aqueous solution. When not the aqueous solution but a crystalline solid raw material such as TMAH pentahydrate is used as a source of the quaternary ammonium hydroxide, the metal impurity content in terms of each metal is preferably no more than 100 mass ppb as well.

The metal impurity content in the first organic solvent used for preparing the raw material mixture liquid is preferably no more than 50 mass ppb, and more preferably no more than 10 mass ppb, in terms of each metal on the basis of the total mass of the first organic solvent. When the impurity content in the first organic solvent, which is commercially available, is high, the first organic solvent is evaporated alone, which can increase the purity.

The first organic solvent used for preparing the raw material mixture liquid is not necessarily an anhydrous solvent. In view of increase of the efficiency of the thin film evaporation, the water content in the first organic solvent used for preparing the raw material mixture liquid is preferably no more than 1 mass %, and more preferably no more than 0.5 mass %, on the basis of the total mass of the first organic solvent.

### (2.2 Step (a): Thin Film Evaporation)

The step (a) is a step of subjecting the raw material mixture liquid to the thin film evaporation by means of a thin film evaporation apparatus, to remove water from the raw material mixture liquid. Thin film evaporation is a method of, in a reduced pressure, forming a thin film of a raw material liquid, heating the thin film, evaporating part of the raw material liquid according to the vapor pressure of the constituents contained in the raw material liquid and cooling and condensing the vapor, and separating the raw material liquid into a distillate and a residue (including a melt). Subjecting the foregoing raw material mixture liquid to the thin film evaporation makes it possible to distill water from the raw material mixture liquid, to recover the organic solvent solution of a quaternary ammonium hydroxide as a residue. Part of the organic solvent may be distilled together with water. Water (and the part of the organic solvent) distilled from the raw material mixture liquid is recovered as a distillate. The thin film evaporation makes it possible to distill water as suppressing thermal decomposition of the quaternary ammonium hydroxide.

### (2.2.1 Thin Film Evaporation Apparatus)

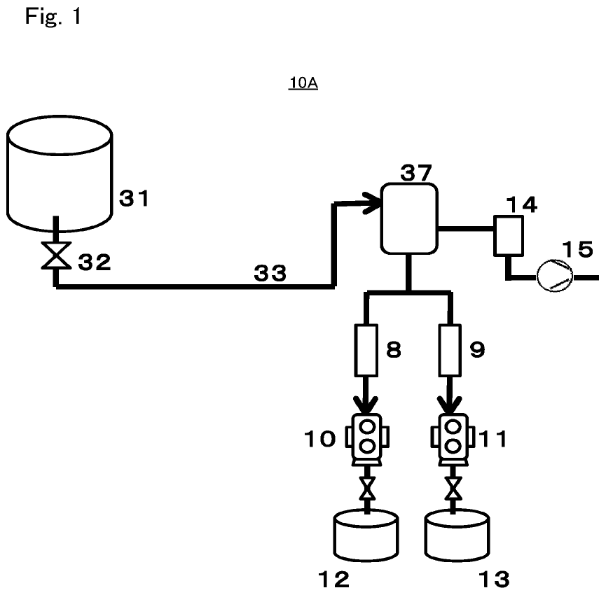

In the step (a), any known thin film evaporation apparatus such as flowing-down-type, centrifugal, rotary, blade type, and climbing thin film evaporation apparatuses may be used as the thin film evaporation apparatus. Among them, a flowing-down-type thin film evaporation apparatus may be especially preferably used. FIG. 1 is an explanatorily schematic view of a thin film evaporation apparatus 10A according to one embodiment (hereinafter may be referred to as “thin film evaporation apparatus 10A” or simply “apparatus 10A”) which may be used in the step (a). The apparatus 10A is a flowing-down-type short-path thin film evaporation apparatus.

The thin film evaporation apparatus 10A comprises a raw material reservoir 31 storing the raw material mixture liquid, an evaporation vessel (evaporation can) 37 where evaporation is actually performed, and a raw material conduit 33 transferring the raw material mixture liquid from the raw material reservoir 31 to the evaporation vessel 37. As shown in FIG. 1, a needle valve 32 is disposed in the middle of the raw material conduit 33. The apparatus 10A further comprises a residue recovery vessel 12 connected to the evaporation vessel 37 and receiving an evaporation residue, a distillate recovery vessel 13 connected to the evaporation vessel 37 and receiving the distillate, a glass conduit for flow rate confirmation 8 and a gear pump (feed pump) (on the residue side) 10 which are disposed in the middle of a flow path introducing the evaporation residue from the evaporation vessel 37 to the residue recovery vessel 12, a glass conduit for flow rate confirmation 9 and a gear pump (feed pump) (on the distillate side) 11 which are disposed in the middle of a flow path introducing the distillate from the evaporation vessel 37 to the distillate recovery vessel 13, a vacuum pump 15 reducing the pressure inside the evaporation vessel 37, and a cold trap 14 disposed in the middle of a flow path from the evaporation vessel 37 to the vacuum pump 15.

The raw material mixture liquid flows out of the raw material reservoir 31, passes through the needle valve 32 and the raw material conduit 33, and flows into the evaporation vessel (evaporation can) 37. The vacuum pump 15, the needle valve 32, and the gear pumps (feed pumps) (on the residue and distillate sides) 10 and 11 operate to maintain a fixed degree of vacuum in the system including the evaporation vessel 37. The raw material mixture liquid in the raw material reservoir 31 spontaneously flows into the raw material conduit 33 via the needle valve 32 due to the differential pressure between the degree of vacuum in the system and atmospheric pressure.

In the thin film evaporation apparatus 10A, liquid-contacting portions in the flow path for the raw material mixture liquid from the raw material reservoir 31 to the evaporation vessel 37, specifically, liquid-contacting portions of the inner surfaces of the raw material reservoir 31 and the raw material conduit 33 (including a liquid-contacting portion of the needle valve 32) are made of resin. The liquid-contacting portions made of resin can suppress elution of metallic materials therefrom. Quaternary ammonium hydroxides available as a raw material inevitably contain water. Generally, water relates to an elution reaction of metallic materials. The liquid-contacting portions in the flow path for the raw material mixture liquid from the raw material reservoir 31 to the evaporation vessel 37 made of resin can shorten the time while the raw material mixture liquid, where the quaternary ammonium hydroxide and water coexist, is in contact with metallic materials, which can suppress such reaction that metallic materials elute into the liquid to be metal impurities in the liquid. In view of further suppression of elution of metallic materials from the liquid-contacting portions, liquid-contacting portions in the flow path from the evaporation vessel 37 to the residue recovery vessel 12 are preferably made of resin as well.

As the resin constituting the liquid-contacting portions, any resin material having durability against an alkaline water and a water-soluble organic solvent may be preferably used. Examples of such a resin material include fluororesins such as polytetrafluoroethylene (PTFE), perfluoroalkoxy alkane (PFA), perfluoroethylene propylene copolymer (FEP), ethylene-tetrafluoroethylene copolymer (ETFE), and polyvinylidene fluoride (PVDF); polyolefin resins such as polyethylene (PE) and polypropylene (PP); thermoplastic resins such as acrylonitrile-butadiene-styrene copolymer synthetic resins (ABS resins), nylon, acrylic resins, acetal resins, and rigid polyvinyl chloride; and thermosetting resins such as melamine resins, furan resins, and epoxy resins. Among them, polyethylene, polypropylene, and fluororesins may be especially preferably used since being easy to process and since the amounts of elution of metal impurities therefrom are small.

A conduit made of resin only may be used as any conduit of a small diameter of which strength as a structural material is not required so much. In contrast, in any conduit of a large diameter, and the raw material reservoir 31 of which strength is required, preferably, structural members are made of a metal material (such as stainless steel) and the liquid-contacting portions are coated with the foregoing resin material. The resin coating over the liquid-contacting portions has only to have a thickness such as not to come off. The thickness may be, for example, preferably approximately 0.5 to 5 mm.

Glass is also known as a material hardly affected by chemicals. A raw material mixture liquid where a highly basic substance such as a quaternary ammonium hydroxide, and water coexist may erode even glass little by little. Thus, resin is preferably used as the material constituting the liquid-contacting portions rather than glass.

Any resin material that is not porous is preferable as the resin material since metal impurities may elute even from the inside of resin when the resin material has a porous structure. The metal impurity content in the resin material constituting the liquid-contacting portions is preferably no more than 1 mass ppm, and more preferably no more than 0.1 mass ppm, in terms of Na, Ca, Al and Fe each on the basis of the total mass of the resin. Such a resin of a high degree of purity is commercially available.

The reason why Na, Ca, Al and Fe are given as the metal impurities in the resin is that: first, these impurities of four metals are typical impurities contaminating resin, and generally, the impurity content of each of these four metals in the resin of no more than 0.1 mass ppm almost always leads to the impurity content of each of other metals in the resin of no more than 0.1 mass ppm; and second, it is not easy to fully grasp the impurity contents of all metals, and it is rare to sufficiently obtain data on commercially available resins from manufacturers. Strictly, the contents of Na, Mg, Al, K, Ca, Ti, Cr, Mn, Fe, Ni, Cu and Zn as described above in the resin are each preferably no more than 1 mass ppm, and more preferably no more than 0.1 mass ppm.

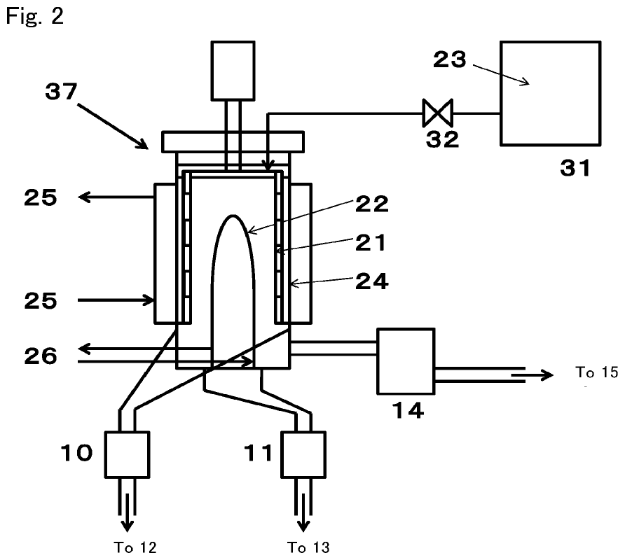

FIG. 2 is a schematically explanatory cross-sectional view of the evaporation vessel 37 in the apparatus 10A. In FIG. 2, the elements already shown in FIG. 1 are given the same reference signs as in FIG. 1, and the description thereof will be omitted. The apparatus 10A comprises the evaporation vessel 37, and a first flow path (raw material conduit 33) introducing the raw material mixture liquid into the evaporation vessel 37 from an upper part of the evaporation vessel 37. The raw material mixture liquid introduced from the first flow path (raw material conduit 33) into the evaporation vessel 37 flows down as a liquid film along the inner wall surface of the evaporation vessel 37. The apparatus 10A further comprises a heating surface 24 arranged in the inner wall surface, and heating the liquid film flowing down along the inner wall surface, a condenser (inside condenser) 22 arranged inside the evaporation vessel 37, and cooling and liquefying a vapor from the liquid film, a second flow path recovering the distillate liquefied by the condenser 22 from the evaporation vessel 37 to the distillate recovery vessel 13, and a third flow path recovering the residue not evaporating but flowing down from the heating surface 24, from the evaporation vessel 37 to the residue recovery vessel 12. The apparatus 10A also comprises a wiper (roller wiper) 21 that is arranged in the evaporation vessel 37 and rotating along the inner wall surface of the evaporation vessel 37. The raw material mixture liquid introduced into the evaporation vessel 37 from the first flow path (conduit 33) is spread on the inner wall surface with the rotating wiper 21, to form the liquid film.

The heating surface 24 is heated by a circulating heat medium 25. The flow rate of a raw material mixture liquid 23 introduced into the evaporation vessel 37 may be adjusted with the needle valve 32 or a flow regulator (not shown). The liquid film is formed on the inner wall of the evaporation vessel 37 with the roller wiper 21, and heat is exchanged on the heating surface 24 arranged in the inner wall surface of the evaporation vessel 37, to evaporate water. At the same time, usually part of the organic solvent evaporates according to the vapor pressure of this organic solvent. The evaporated water and organic solvent are condensed in the condenser (inside condenser) 22 arranged in the vicinity of the center of the evaporation vessel 37 and separated from the liquid film, to be the distillate. The condenser 22 is cooled by a circulating refrigerant 26.

Generally, the inner wall of the evaporation vessel 37 is preferably constituted of a metallic material of high corrosion resistance such as stainless steel in view of comprehensive material properties of heat resistance, anti-wear properties, corrosion resistance, thermal conductivity, strength, etc. It may be also considered that the inner wall of the evaporation vessel 37 is formed of a resin member, or a metallic member coated with resin in view of further suppression of elution of metal impurities. When the inner wall of the evaporation vessel 37 is also formed of a resin member, or a member coated with resin, the efficiency of the heat exchange between the liquid film and the heat medium 25 on the heating surface 24 lowers, which makes it necessary to control the heating surface 24 so that the heating surface 24 has a higher temperature, which may result in progress of thermal decomposition of the quaternary ammonium hydroxide during the thin film evaporation. The roller wiper 21 rotates inside the evaporation vessel 37, which may cause the resin to come off from the inner wall of the evaporation vessel 37 formed of a resin member, or a member coated with resin when the roller wiper 21 comes into contact with the inner wall of the evaporation vessel 37, to mix resin pieces into the recovered residue.

Even if the inner wall of the evaporation vessel 37 is formed of a metallic material, the metal impurity content in the obtained composition (residue) does not deteriorate so much. The reason for this is not fully understood, but the following three are considered: (1) the residence time of the liquid film on the inner wall surface of the evaporation vessel is several seconds to several minutes, which are short for metal impurities to elute; (2) water is necessary for such reaction that a metallic material elutes in an alkaline water, but in the thin film evaporation, water is almost removed from the liquid film in a short time, so that the time while the condition for elution of metal impurities is satisfied is very short; and (3) the composition obtained by the production method according to the present invention usually has a viscosity higher than that of the water-soluble organic solvent in the composition; the raw material mixture liquid has a relatively high viscosity according to the viscosity of the water-soluble organic solvent and the concentration of the quaternary ammonium hydroxide, and this viscosity further increases by distillation of water; that is, it is considered that when the raw material mixture liquid passes along the heating surface 24 of the evaporation vessel 37, most of water is lost in a short time on the interface between the heating surface 24 and the liquid film and the viscosity of the liquid increases, so that a flow stirring the liquid is hardly generated inside the liquid film, which makes it difficult for water to be in contact with the heating surface 24, to, as a result, suppress elution of metal impurities.

A roller wiper made of resin may be used as the roller wiper 21. Preferably, a reinforcing member made of glass fiber or the like is not incorporated in the resin material constituting the roller wiper 21. The roller wiper 21 continues to be in contact with the raw material mixture liquid and the liquid film during the thin film evaporation, which may lead to elution of metal impurities in the glass fiber such as Al and Ca into the liquid if the glass fiber is contained in the resin constituting the roller wiper 21. The roller wiper 21 in contact with the inner wall surface in the evaporation vessel 37 may lead to contamination of fragments of the glass fiber contained in the resin constituting the roller wiper 21 and fine particles generated from the inner wall surface into the residue.

Preferred examples of the resin material constituting the roller wiper 21 include resins having heat resistance and relatively high strength such as: general-purpose engineering plastics including polyacetal (POM), polyamide (PA), polycarbonate (PC), modified polyphenylene ether (m-PPE), polybutylene terephthalate (PBT), ultrahigh molecular weight polyethylene (UHPE), and syndiotactic polystyrene (SPS); and super engineering plastics such as polyether ether ketone (PEEK), polyimide (PI), polyetherimide (PEI), and fluororesins. Among them, PEEK, PI, fluororesins, etc. may be preferably used in view of heat resistance, strength, purity, etc.

The distillate condensed by the condenser 22 is introduced and recovered into the distillate recovery vessel 13 through the second flow path including the gear pump (feed pump) (on the distillate side) 11. A vapor not condensed is captured and recovered in the cold trap 14. The residue from which water is distilled and which flows down from the heating surface 24 is introduced and recovered into the residue recovery vessel 12 through the third flow path including the gear pump (feed pump) (on the residue side) 10.

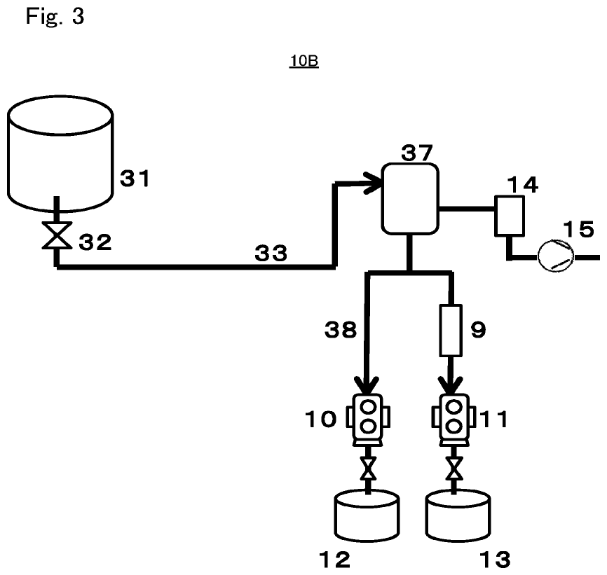

In the thin film evaporation apparatus 10A (FIG. 1), for the purpose of, for example, confirming the flow of the liquid after evaporation, the glass conduit for flow rate confirmation on the residue side 8 (hereinafter may be referred to as “glass conduit 8”) is disposed in the third flow path recovering the residue, and the glass conduit for flow rate confirmation on the distillate side 9 (hereinafter may be referred to as “glass conduit 9”) is disposed in the second flow path recovering the distillate. The glass conduits 8 and 9 are not always necessary. Rather, since made of glass, the glass conduits 8 and 9 may be sources of contamination (sources of elution of metal impurities). In view of further reduction of the metal impurity content in the composition (residue) to be produced, for example, a thin film evaporation apparatus 10B (FIG. 3) such that the glass conduit 8 is removed from the third flow path of the thin film evaporation apparatus 10A may be preferably used instead of the thin film evaporation apparatus 10A (FIG. 1).

The thin film evaporation apparatus 10A (FIG. 1) comprises the gear pump (feed pump) (on the residue side) 10 disposed in the middle of the third flow path introducing the residue from the evaporation vessel 37 to the residue recovery vessel 12, and the gear pump (feed pump) (on the distillate side) 11 disposed in the middle of the second flow path introducing the distillate from the evaporation vessel 37 to the distillate recovery vessel 13 as elements for keeping airtightness in the system including the inside of the evaporation vessel 37. The gear pumps (feed pumps) 10 and 11 are feed pumps to push the liquids on the residue side and the distillate side toward the recovery vessels 12 and 13 as keeping airtightness in the system. The material of each component (such as a casing and a gear) used for the liquid-contacting portions of these feed pumps that also serve as airtightness may be a metallic material having sufficient corrosion resistance (such as stainless steel). The reason for this is the same as the reason why the inner wall of the evaporation vessel does not need to be coated with resin. That is, it is considered that: the water content of the residue with which the liquid-contacting portion of the feed pump on the residue side 10 is in contact is sufficiently low, and the time while the residue is in contact with the liquid-contacting portion of the feed pump 10 is sufficiently short, which hardly lead to elution of metal impurities from the liquid-contacting portion of the feed pump 10 into the residue even if the liquid-contacting portion of the feed pump 10 is made of a metallic material such as stainless steel, for example. In view of further reduction of the metal impurity content in the composition to be produced, a feed pump having a liquid-contacting portion made of resin such as an engineering plastic or a super engineering plastic may be used as the feed pump on the residue side 10.

Examples of the vacuum pump 15 include known vacuum pumps such as an oil rotary pump, an oil diffusion pump, a cryopump, a swing piston vacuum pump, a mechanical booster pump, a diaphragm pump, a roots type dry pump, a screw dry pump, a scroll dry pump, and a vane dry pump. As the vacuum pump 15, one vacuum pump may be used alone, or a plurality of vacuum pumps may be used in combination.

The cold trap 14 plays a role so that the vapor not condensed in the condenser 22 is condensed or solidified into a liquid or a solid, to prevent the evaporated water or organic solvent from reaching the vacuum pump 15, and to prevent vaporized oil or oil mist from flowing into the evaporation vessel 37 side from the vacuum pump 15 such as an oil rotary pump and contaminating the inside of the system. As the cold trap 14, any known cold trap device may be used. The cold trap 14 may be cooled using, for example, dry ice, a coolant obtained by mixing dry ice with an organic solvent (such as an alcohol, acetone and hexane), liquid nitrogen, and a circulating refrigerant.

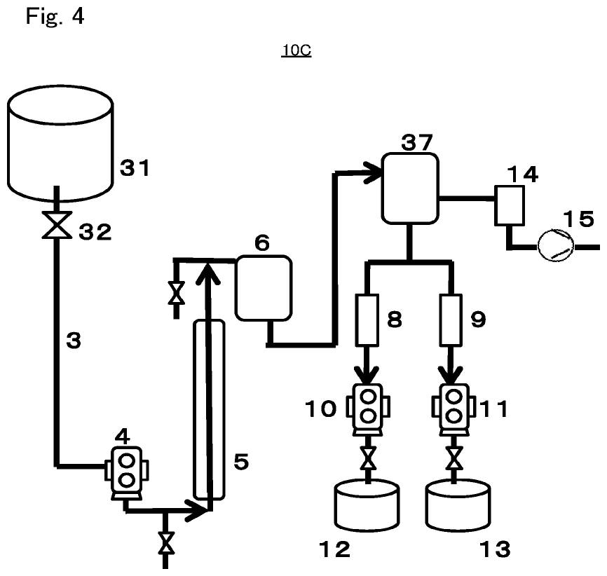

The thin film evaporation apparatuses 10A (FIG. 1) and 10B (FIG. 3) each comprising the feed pumps 10 and 11 only on the downstream side of the evaporation vessel 37 have been described as examples. The thin film evaporation apparatus may further comprise the feed pump on the upstream side of the evaporation vessel 37 as well. FIG. 4 is an explanatorily schematic view of a thin film evaporation apparatus 10C (hereinafter may be simply referred to as “apparatus 10C”) according to such another embodiment. In FIG. 4, the elements already shown in FIGS. 1 to 3 are given the same reference signs as in FIGS. 1 to 3, and the description thereof will be omitted. The thin film evaporation apparatus 10C is different from the thin film evaporation apparatus 10A (FIG. 1) in that the thin film evaporation apparatus 10C has a raw material conduit 3 instead of the raw material conduit 33 introducing the raw material mixture liquid from the raw material reservoir 31 to the evaporation vessel 37, and further has a raw material gear pump 4, a preheater 5 and a degasser 6 in the middle of the raw material conduit 3 on the downstream side of the needle valve 32, in the order mentioned from the upstream side. In the apparatus 10C, liquid-contacting portions in the flow path for the raw material mixture liquid from the raw material reservoir 31 to the evaporation vessel 37, that is, liquid-contacting portions of the raw material conduit 3 (including the needle valve 32), the raw material gear pump 4, the preheater 5 and the degasser 6 are made of a resin material. It generally causes an increase in apparatus costs that all the liquid-contacting portions of the raw material gear pump 4, the preheater 5 and the degasser 6 are constituted of a resin material. Thus, a thin film evaporation apparatus not comprising the raw material gear pump 4, the preheater 5 or the degasser 6 like the apparatuses 10A and 10B may be preferably employed.

The thin film evaporation apparatuses 10A (FIG. 1), 10B (FIG. 3) and 10C (FIG. 4) each comprising a needle valve as the valve 32 have been described as examples. It is not always necessary that the valve 32 is a needle valve. Any other known valves such as a diaphragm valve, a butterfly valve, a ball valve and a gate valve may be employed as the valve 32 instead of a needle valve.

Examples of a commercially available thin film evaporation apparatus that may be used in the step (a) include short-path evaporation apparatus (manufactured by UIC GmbH); WIPRENE (registered trademark) and EXEVA (registered trademark) (both manufactured by Kobelco Eco-Solutions Co., Ltd.); Kontro and Sevcon (registered trademark) (both manufactured by Hitachi Plant Mechanics Co., Ltd.); Viscon and Filmtruder (both manufactured by Buss-SMS-Canzler GmbH, available from KIMURA CHEMICAL PLANTS CO., LTD.); EVA reactor, Hi-U Brusher, and Wall Wetter (all manufactured by Kansai Chemical Engineering Co., Ltd.); NRH (manufactured by Nitinan Kikai Kabushiki-kaisha); and EVAPOR (registered trademark) (manufactured by OKAWARA MFG. CO., LTD.). A flowing-down-type thin film evaporation apparatus is preferably used in view of enhancement of efficiency of evaporation since a quaternary ammonium hydroxide heated for a long time decomposes. In the same point of view, a short-path thin film evaporation apparatus may be preferably used, and a flowing-down-type short-path thin film evaporation apparatus may be particularly preferably used.

In the present description, a flowing-down-type thin film evaporation apparatus means a thin film evaporation apparatus such that a thin film of a liquid introduced into the evaporation vessel (liquid film) is formed on the heating surface inside the evaporation vessel (for example, by a rotating blade or the like), and evaporation is performed while the liquid film is made to flow down along the heating surface. A short-path thin film evaporation apparatus (short-path evaporation apparatus) is a thin film evaporation apparatus that has been developed to enhance separation performance based on the technical idea of molecular evaporation as a starting point. In a short-path evaporation apparatus, a condenser is arranged inside a cylindrical evaporation vessel so that a cooling surface of the condenser faces a heating surface of the evaporation vessel. Evaporation using a short-path evaporation apparatus (short-path evaporation) is often performed under a pressure of approximately medium vacuum (order of 10⁻¹ to 10² Pa).

When the foregoing thin film evaporation apparatus that is commercially available is used, it is preferable to use an apparatus that is modified so that the liquid-contacting portions on the upstream side of the evaporation vessel are made of resin.

### (2.2.2 Evaporation Conditions)

The properties of the organic solvent solution of a quaternary ammonium hydroxide obtained by the thin film evaporation may be mainly influenced by the temperature of the raw material mixture liquid right before the raw material mixture liquid enters the evaporation vessel 37 (first temperature), the temperature of the heating surface 24 of the evaporation vessel 37 (second temperature), and the degree of vacuum in the system.

The temperature of the raw material mixture liquid right before the raw material mixture liquid enters the evaporation vessel 37 (first temperature) is preferably no more than 70°, and more preferably no more than 60° C. The first temperature at this upper limit or less can further reduce elution of metal impurities from the evaporation vessel 37 when the raw material mixture liquid having a high water content is in contact with the inner wall surface of the evaporation vessel 37. The first temperature is preferably no less than 5° C., and more preferably no less than 15° C. The first temperature at this lower limit or more can suppress formation of precipitation containing the quaternary ammonium hydroxide, and can further enhance efficiency of evaporation.

The temperature of the heating surface 24 (second temperature) is preferably higher than the first temperature, and preferably 60 to 140° C., and more preferably 70 to 120° C. The second temperature at this lower limit or more can further enhance efficiency of evaporation, to quickly reduce the water content in the liquid film, which can further reduce elution of metal impurities from the evaporation vessel 37. The second temperature at this upper limit or less can reduce evaporation of the organic solvent, and can further reduce elution of metal impurities from the evaporation vessel 37. In the present description, “temperature of the heating surface” of the thin film evaporation apparatus means the temperature of a heat source by which the liquid film is heated.

The degree of vacuum in the system (the degree of vacuum from the inside of the evaporation vessel 37 or from the evaporation vessel 37 to a portion in front of the vacuum pump) is preferably no more than 600 Pa, more preferably no more than 550 Pa, and further preferably no more than 400 Pa, and in one embodiment, may be no more than 200 Pa. The degree of vacuum in the system at this upper limit or less can enhance efficiency of evaporation to quickly reduce the water content in the liquid film, which can further reduce elution of metal impurities from the evaporation vessel 37. The lower limit of the degree of vacuum is not particularly limited, but may be no less than 0.1 Pa in one embodiment, and no less than 1 Pa in another embodiment. The degree of vacuum in the system at this lower limit or more makes it easy to avoid blockage of a conduit in the exhaust system due to the evaporated that condense or solidify in the cold trap 14. The degree of vacuum in the system may be measured using a pressure measuring instrument (not shown) disposed in the middle of a conduit in the exhaust system which connects the evaporation vessel 37 and the vacuum pump 15, such as a manometer and a vacuum gauge. In one embodiment, a pressure measuring instrument may be disposed between the cold trap 14 and the vacuum pump 15.

A preferred feed rate of the raw material mixture liquid to the evaporation vessel 37 may vary depending on the scale of the thin film evaporation apparatus. Too high a feed rate leads to deterioration of efficiency of evaporation, and too low a feed rate leads to deterioration of productivity. If the evaporation conditions such as the temperature of the heating surface 24 and the degree of vacuum in the evaporation vessel 37 are the same, a larger heat transfer area of the thin film evaporation apparatus (area of the heating surface 24) can increase the feed rate more. For example, use of the thin film evaporation apparatus having a heat transfer area of 0.1 m² can lead to a preferred feed rate of 1 to 10 kg/hour. The temperature of the raw material mixture liquid right before the raw material mixture liquid enters the evaporation vessel 37 (first temperature), the temperature of the heating surface 24 (second temperature), and the degree of vacuum in the system (degree of vacuum from the inside of the evaporation vessel 37 or from the evaporation vessel 37 to a portion in front of the vacuum pump) within the above ranges can lead to a feed rate per unit area of the heating surface 24 of, for example, 10 to 100 kg/hour·m².

The step (a) is performed, which makes it possible to evaporate and remove water from the raw material mixture liquid, to obtain the organic solvent solution of a quaternary ammonium hydroxide.

### (2.3 Step (b): Washing Step)

Preferably, the solution production method according to the present invention further comprises, prior to the step (a), washing the liquid-contacting portions in the flow path for the raw material mixture liquid from the material reservoir 31 to the evaporation vessel 37 (for example, in the apparatus 10A, the liquid-contacting portion of the inner surface of the material reservoir 31, and the liquid-contacting portion of the raw material conduit 33 (including the liquid-contacting portion of the needle valve 32)) with a solution comprising the foregoing quaternary ammonium hydroxide (hereinafter, may be referred to as “step (b)). Preferred examples of cleaning solutions used for washing in the step (b) include solutions containing the foregoing quaternary ammonium hydroxide such as the aqueous quaternary ammonium hydroxide solution, which is used as part of the raw material, and the raw material mixture liquid. Among them, a solution containing the quaternary ammonium hydroxide same as the quaternary ammonium hydroxide contained in the raw material mixture liquid may be particularly preferably used as the cleaning solution. The metal impurity content in the solution containing the quaternary ammonium hydroxide (cleaning solution) is preferably no more than 0.05 mass ppm, more preferably no more than 0.02 mass ppm, and further preferably no more than 0.01 mass ppm, in terms of Na, Mg, Al, K, Ca, Ti, Cr, Mn, Fe, Ni, Cu, and Zn each on the basis of the total mass of the solution.

For example, the cleaning solution is made to flow on resin portions of the liquid-contacting portions for approximately 10 minutes to 2 hours, or the cleaning solution is stored and held in the resin portions of the liquid-contacting portions, which makes it possible to wash the liquid-contacting portions. Carrying out the step (b) prior to the step (a) leads to reduction or removal of metal impurities in an elutable state from the surface of the resin, which can further reduce metal impurities eluting from the liquid-contacting portions during the thin film evaporation. In one preferred embodiment, the liquid-contacting portions may be further washed (rinsed) in a short time with water having a very low metal impurity content such as ultrapure water and pure water after washed with the aqueous quaternary ammonium hydroxide solution or the raw material mixture liquid. The step (b) according to such an embodiment can further reduce the amount of elution of metal impurities from the liquid-contacting portions in the step (a). When, for example, it is apparent that elutable metal impurities have already been reduced or removed from the surface of the resin of the liquid-contacting portions, a method for producing the organic solvent solution of a quaternary ammonium hydroxide not comprising the step (b) may be employed.

Preferably, an acid aqueous solution is not used for washing the liquid-contacting portions. An acid aqueous solution in contact with the liquid-contacting portions easily leads to an anion contained in the acid aqueous solution remaining on the surface of the resin, and it takes a long time to wash and remove the anion with ultrapure water, pure water, or the like. Therefore, the liquid-contacting portions are preferably washed using the solution containing the quaternary ammonium hydroxide (and optionally water having a very low metal impurity content such as pure water and ultrapure water).

### (2.4. Properties of Organic Solvent Solution of Quaternary Ammonium Hydroxide)

### (2.4.1 Content of Quaternary Ammonium Hydroxide)

In one embodiment, the content of the quaternary ammonium hydroxide in the organic solvent solution of a quaternary ammonium hydroxide obtained by the solution production method according to the present invention (hereinafter may be simply referred to as a “solution”) may be preferably no less than 5.0 mass %, and more preferably no less than 8.0 mass %, on the basis of the total mass of the solution. The content of the quaternary ammonium hydroxide in the solution at the above lower limit or more makes it possible to save the distribution cost for the solution. The upper limit of this content is not particularly limited, but may be no more than 72 mass % in one embodiment, and no more than 55 mass % in another embodiment. The content of the quaternary ammonium hydroxide in the solution at the above upper limit or less results in suppression of improvement in viscosity of the solution, which makes it easy to, e.g., handle, feed, and mix the solution when the solution is used.

The concentration of the quaternary ammonium hydroxide in the solution can be accurately measured with a potentiometric titration apparatus, liquid chromatography, etc. These measuring means may be used alone, or may be used in combination.

In one embodiment, the content of the quaternary ammonium hydroxide in the solution may be 2.38 to 25.0 mass %. In one preferred embodiment, TMAH may be used as the quaternary ammonium hydroxide. The TMAH content in the solution may be 2.38 to 25.0 mass % on the basis of the total mass of the solution.

### (2.4.2 Water Content)

The water content in the solution obtained by the solution production method according to the present invention is no more than 1.0 mass %, preferably no more than 0.5 mass %, and more preferably no more than 0.3 mass %, on the basis of the total mass of the solution. The water content in the solution at the above upper limit or less can enhance the removing performance for modified photoresists and residue of ashed photoresists, and can reduce the corrosivity to metallic materials and inorganic substrate materials. The lower limit of the water content in the solution is not particularly limited, but may be, for example, no less than 0.05 mass %.

The water content in the solution may be preferably measured by the same method as described in the section 1.4 in connection with the treatment liquid composition for semiconductor production according to the first aspect of the present invention.

The ratio of the water content in the solution (unit: mass %) to the content of the quaternary ammonium hydroxide in the solution (unit: mass %) (water content/content of the quaternary ammonium hydroxide) is preferably no more than 0.42, more preferably no more than 0.21, and further preferably no more than 0.10. This ratio at the above upper limit or less makes it possible to maintain or improve the removing performance for modified photoresists and residue of ashed photoresists, and at the same time to further reduce the corrosivity to metallic materials and inorganic substrate materials. The lower limit of this ratio is not particularly limited, but may be, for example, no less than 0.0007.

### (2.4.3 Impurity Content)

The metal impurity content in the solution obtained by the solution production method according to the present invention is no more than 100 mass ppb, preferably no more than 50 mass ppb, and more preferably no more than 20 mass ppb, in terms of Na, Mg, Al, K, Ca, Ti, Cr, Mn, Fe, Ni, Cu, and Zn each on the basis of the total mass of the solution. In the present description, the metal impurity content in the solution means the total content of metal elements in the metal impurities regardless of whether each of the metal elements is a zero-valent metal or in the form of a metal ion.

The chlorine impurity (Cl) content in the solution is no more than 100 mass ppb, preferably no more than 80 mass ppb, and more preferably no more than 50 mass ppb, on the basis of the total mass of the solution. In the present description, the chlorine impurity content in the solution means the total content of a chlorine element. In the solution, chlorine impurities are usually present in the form of a chloride ion (Cl⁻).

The metal impurity content in the solution can be measured with a microanalyzer such as an inductively coupled plasma mass spectrometer (ICP-MS). The chlorine impurity content can be measured by a microanalyzer using ion chromatography etc.

The ratio of the metal impurity content in the solution (unit: mass ppb) to the content of the quaternary ammonium hydroxide in the solution (unit: mass %) (metal impurity content/content of the quaternary ammonium hydroxide) is preferably no more than 42, more preferably no more than 21, and further preferably no more than 10, in terms of each of the foregoing metal elements. This ratio at the above upper limit or less makes it possible to maintain or improve the removing performance for modified photoresists and residue of ashed photoresists, and at the same time to further increase yields of semiconductor devices. The lower limit of this ratio is not particularly limited, but the lower the more preferable. The lower limit thereof may be, for example, no less than 0.0001 in view of, for example, the quantitative limit of a measurement device for metal impurities.

The ratio of the chlorine impurity content in the solution (unit: mass ppb) to the content of the quaternary ammonium hydroxide in the solution (unit: mass %) (chlorine content/content of the quaternary ammonium hydroxide) is preferably no more than 42, more preferably no more than 34, and further preferably no more than 21. This ratio at the above upper limit or less makes it possible to maintain or improve the removing performance for modified photoresists and residue of ashed photoresists, and at the same time to further increase yields of semiconductor devices. The lower limit of this ratio is not particularly limited, but the lower the more preferable. The lower limit thereof may be, for example, no less than 0.001 in view of, for example, the quantitative limit of a measurement device for chlorine impurities.

### (2.4.4 Use)

For example, the solution obtained by the solution production method according to the present invention may be preferably used as a chemical liquid used in the production process of semiconductor devices, such as developers for photoresists, strippers and cleaning solutions for modified photoresists, and silicon etchants. In addition, this solution may be preferably used as a concentrated liquid that is a raw material for producing the foregoing chemical liquid. For example, the solution obtained by the production method according to the present invention is diluted with the first organic solvent, or the second organic solvent, or any combination thereof, which makes it possible to obtain a chemical liquid having a desired concentration of the quaternary ammonium hydroxide.

Water is added to the solution obtained by the solution production method according to the present invention, which makes it possible to produce various chemical liquids each having a controlled water content. That is, the organic solvent solution obtained by the solution production method according to the present invention may be used as a raw material for producing a chemical liquid having a controlled water content. A solution having a composition such that the concentrations of the quaternary ammonium hydroxide and the organic solvent are within desired ranges is not always obtained only by diluting any quaternary ammonium hydroxide that is commercially available on an industrial scale as described in the section 2.1.3 with the organic solvent. As a raw material for obtaining the solution of the quaternary ammonium hydroxide having such a composition, the solution obtained by the solution production method according to the present invention is useful.

For example, an etchant such as a silicon etchant may be required of control of the etching rate according to the water content. In such use, it is required to strictly control the water content in the chemical liquid. A solution having a strictly controlled water content can be obtained by adding water of a high degree of purity such as ultrapure water to the solution obtained by the solution production method according to the present invention. Water may be added in such a purpose so that, for example, the water content in the solution is preferably 1.0 to 40 mass %, more preferably 2.0 to 30 mass %, and further preferably 3.0 to 20 mass %, on the basis of the total mass of the solution. The water content that the solution after water is added should have is determined by, for example, a desired etching rate. In order to adjust both the water content and the concentration of the quaternary ammonium hydroxide, the organic solvent described in the sections 1.2 and 1.3 (the first organic solvent, or the second organic solvent, or any combination thereof) may be added together with water.

### <3. Method for Producing Treatment Liquid Composition for Semiconductor Production>

A method for producing a treatment liquid composition for semiconductor production according to the third aspect of the present invention (hereinafter may be referred to as “composition production method”) is a method for producing the treatment liquid composition for semiconductor production according to the first aspect of the present invention comprising (i) obtaining an organic solvent solution of a quaternary ammonium hydroxide by the solution production method according to the second aspect of the present invention (hereinafter may be referred to as “step (i)”), (ii) knowing the concentration of the quaternary ammonium hydroxide in the organic solvent solution (hereinafter may be referred to as “step (ii)”), and (iii) adding the organic solvent to the solution, to adjust the concentration of the quaternary ammonium hydroxide in the solution (hereinafter may be referred to as “step (iii)”).

### (3.1 Step (i): Solution Production Step)

The step (i) is a step of obtaining an organic solvent solution of a quaternary ammonium hydroxide by the solution production method according to the second aspect of the present invention, and details thereof are as described in the section 2.

### (3.2 Step (ii): Concentration Knowing Step)

The step (ii) is a step of knowing the concentration of the quaternary ammonium hydroxide in the solution obtained in the step (i). The concentration of the quaternary ammonium hydroxide in the solution may be preferably measured by the method same as described in the section 2.4.1 in connection with the solution production method according to the second aspect of the present invention. If there are the results of the production of the organic solvent solution of the quaternary ammonium hydroxide by the solution production method according to the second aspect of the present invention under the same conditions where the step (i) is carried out (composition of the raw material mixture liquid and evaporation conditions), and the measurement of the concentration of the quaternary ammonium hydroxide in the obtained solution in the past, the concentration of the quaternary ammonium hydroxide in the solution measured in the past operation results may be regarded as the concentration of the quaternary ammonium hydroxide in the solution obtained in the step (i).

The concentration of the quaternary ammonium hydroxide in the solution can be accurately measured with a commercially available measurement device such as a potentiometric titration apparatus and a liquid chromatograph. These measuring means may be used alone, or may be used in combination. As a sample used for the measurement, a sample collected from the solution may be used as it is, or a diluted sample obtained by accurately diluting a sample collected from the solution with a solvent (such as water) may be used.

A potentiometric titration apparatus is an apparatus for measurement according to the potentiometric method specified in JIS K0113. A potentiometric titration apparatus capable of automatic measurement is commercially available, and may be preferably used. The potentiometric method is an electrochemical measurement method such that the equivalent point of volumetric analysis is determined based on the change in the electrode potential difference between an indicator electrode and a reference electrode in response to the concentration (activity) of a target constitution in a solution to be titrated.

A potentiometric titration apparatus includes a titration tank where a solution to be titrated is put, a burette for adding a standard solution to the titration tank, an indicator electrode and a reference electrode to be put in the solution, and a potentiometer for measuring the potential difference between both electrodes. Measurement using a potentiometric titration apparatus is performed, for example, as follows. A solution to be titrated is put in the titration tank, a proper indicator electrode and reference electrode are inserted therein, and the potential difference between both electrodes is measured by the potentiometer. Next, a predetermined amount of a standard solution is dropped from the burette into the titration tank and stirred well to react the standard solution with the solution to be titrated, and thereafter the potential difference between the both electrodes is measured. This operation is repeated, and the potential differences between the both electrodes corresponding to the amounts of the added standard solution are recorded, so that a potential difference-standard solution amount curve (hereinafter may be referred to as “potentiometric titration curve”) is obtained. In the obtained potentiometric titration curve, the amount of the added standard solution corresponding to the point at which the potential difference sharply changes is obtained, which makes it possible to determine the end point of the titration. The concentration of the target constitution in the solution to be titrated can be calculated from the amount and the concentration of the added standard solution dropped until the end point of the titration, the reaction molar ratio of the titration reaction, etc. When the concentration of the quaternary ammonium hydroxide is measured, an acid such as sulfuric acid and hydrochloric acid (e.g., no more than 1.0 N) is usually used as the standard solution. When the solution contains only one quaternary ammonium hydroxide, the concentration of the quaternary ammonium hydroxide in the solution (mol/L) can be measured quickly and conveniently by the potentiometric method. When the solution contains two or more quaternary ammonium hydroxides, the total concentration of the quaternary ammonium hydroxide in the solution (mol/L) can be also measured quickly and conveniently by the potentiometric method.

When the mixing ratio of the quaternary ammonium hydroxide in the solution containing two or more quaternary ammonium hydroxides is unknown, the mixing molar ratio of the quaternary ammonium hydroxide in the solution can be accurately measured by using liquid chromatography. For example, standard samples each containing a quaternary ammonium hydroxide of a known concentration are prepared (the concentration of a quaternary ammonium hydroxide in each of the standard samples (mol/L) can be accurately measured by the potentiometric method); mixtures obtained by mixing the standard samples at a plurality of different mixing ratios are subjected to measurement by liquid chromatography, the ratio of the peak strength in each chromatogram is plotted with respect to the mixing ratio, to draw a calibration curve; the organic solvent solution of a quaternary ammonium hydroxide containing two or more quaternary ammonium hydroxides of an unknown mixing ratio is subjected to measurement by liquid chromatography; and the mixing molar ratio of the quaternary ammonium hydroxides in the solution can be obtained from the ratio of the peak strength in a chromatogram, using the calibration curve. The total concentration of the quaternary ammonium hydroxide in the solution (mol/L) can be measured by the potentiometric method as described above. Thus, the concentration of each of the quaternary ammonium hydroxides in the solution containing two or more quaternary ammonium hydroxides can be accurately measured by the combination of measurement by the potentiometric method and measurement by liquid chromatography.

The mixing ratio of the quaternary ammonium hydroxides in the raw material mixture liquid is often known at the time point when the raw material mixture liquid containing two or more quaternary ammonium hydroxides is prepared. Further, a quaternary ammonium hydroxide does not evaporate even if the raw material mixture liquid is subjected to the thin film evaporation in the step (i). Therefore, actually, it is not often the case that measurement by liquid chromatography is performed.

The above described measurement method is also applicable to measurement of the concentration of the quaternary ammonium hydroxide in the composition according to the first aspect of the present invention, and measurement of the concentration of the quaternary ammonium hydroxide in the raw material mixture liquid.

(3.3 Step (iii): Diluting Step)

The step (iii) is a step of adding an organic solvent to the solution obtained in the step (i), to adjust the concentration of the quaternary ammonium hydroxide in the solution. That is, the step (iii) is a step of diluting the solution obtained in the step (i) with an organic solvent.

### (3.3.1 Dilution Solvent)

As an organic solvent used in the step (iii) (hereinafter may be referred to as “dilution solvent”), any organic solvent that may be mixed with the first organic solvent contained in the solution obtained in the step (i) may be used. Examples of a preferred dilution solvent include water-soluble organic solvents each having a plurality of hydroxy groups (first organic solvent) described in the section 1.2 in connection with the composition according to the first aspect of the present invention, and a preferred embodiment thereof is also the same as described above. In one embodiment, the water-soluble organic solvent same as the first organic solvent contained in the solution obtained in the step (i) may be particularly preferably used as the dilution solvent.

As described in the section 1.3 in connection with the composition according to the first aspect of the present invention, the composition according to the first aspect of the present invention may further comprise any organic solvent (second organic solvent) other than a water-soluble organic solvent having a plurality of hydroxy groups, in addition to the water-soluble organic solvent (first organic solvent) having a plurality of hydroxy groups, as the solvent. In order to obtain the composition containing such a second organic solvent, the first organic solvent and the second organic solvent may be used in combination as the dilution solvent in the step (iii). Examples of the second organic solvent include organic solvents described in the section 1.3 as the second organic solvent, and a preferred embodiment thereof is also the same as described above.

In the step (iii), the amount of each added organic solvent that constitutes the dilution solvent may be determined so that the concentration of each constitution in the composition to be produced is within a desired range.

The water content in the dilution solvent is no more than 1.0 mass %, preferably no more than 0.5 mass %, and more preferably no more than 0.3 mass %, on the basis of the total mass of the dilution solvent. The water content in the dilution solvent at the above upper limit or less can enhance the removing performance of the obtained composition for modified photoresists and residue of ashed photoresists, and can reduce the corrosivity to metallic materials and inorganic substrate materials, when the obtained composition is used as a stripper and a cleaning solution. The lower limit of the water content in the dilution solvent is not particularly limited, but may be, for example, no less than 0.05 mass %.

The metal impurity content in the dilution solvent is no more than 100 mass ppb, preferably no more than 50 mass ppb, and more preferably no more than 20 mass ppb, in terms of Na, Mg, Al, K, Ca, Ti, Cr, Mn, Fe, Ni, Cu and Zn each on the basis of the total mass of the dilution solvent. In the present description, the metal impurity content in the dilution solvent means the total content of metal elements in the metal impurities regardless of whether each of the metal elements is a zero-valent metal or in the form of a metal ion.

The chlorine impurity (Cl) content in the dilution solvent is no more than 100 mass ppb, preferably no more than 80 mass ppb, and more preferably no more than 50 mass ppb, on the basis of the total mass of the dilution solvent. In the present description, the chlorine impurity content in the dilution solvent means the total content of a chlorine element. In the dilution solvent, chlorine impurities are usually present in the form of a chloride ion (Cl⁻).

The metal impurity content in the dilution solvent can be measured with a microanalyzer such as an inductively coupled plasma mass spectrometer (ICP-MS). The chlorine impurity content can be measured by a microanalyzer using ion chromatography etc.

### (3.3.2 Dilution Conditions)

In the step (iii), the amount of the dilution solvent added to the solution obtained in the step (i) may be such an amount that the composition according to the first aspect of the present invention is obtained. Such an amount can be determined from the concentration of the quaternary ammonium hydroxide in the solution obtained in the step (i).

The steps (i) to (iii) are performed, which makes it possible to preferably produce the treatment liquid composition for semiconductor production according to the first aspect of the present invention.

### (3.4 Production of Other Chemicals)

The method for producing the composition according to the present invention is also applicable to production of a composition (chemical liquid) modified so that the water content is more than 1.0 mass % on the basis of the total mass of the composition, such as an etchant described in the section 2.4.4. In the step (iii) (diluting step) described in the section 3.3, further addition of a necessary amount of water (e.g., ultrapure water) makes it possible to produce a composition modified so that the water content is more than 1.0 mass % on the basis of the total mass of the composition. In such a modified production method, the water content of the organic solvent (dilution solvent) used in the step (iii) (diluting step) may be more than 1.0 mass % as long as the concentrations of metal impurities and chlorine impurities thereof are within the ranges described in the section 3.3.1.

### EXAMPLES

Hereinafter the present invention will be described in more detail using examples and comparative examples. The following examples are merely examples for explaining the present invention, and the present invention is not limited to these examples.

### (Measurement Method)

In each of the examples and comparative examples, the concentration of a quaternary ammonium hydroxide in a solution was measured by potentiometric titration using an automatic potentiometric titration apparatus AT-610 (manufactured by KYOTO ELECTRONICS MANUFACTURING CO., LTD.).

The water contents in the obtained solutions were obtained by correction of the measurements by Karl Fischer titration using a calibration curve. The water contents were measured by Karl Fischer titration using a Karl Fischer titrator MKA-510 (manufactured by KYOTO ELECTRONICS MANUFACTURING CO., LTD.). The water contents were measured by gas chromatography (hereinafter may be simply referred to as “GC”) using a gas chromatograph GC-2014 manufactured by SHIMADZU CORPORATION (column: DB-WAX manufactured by Agilent Technologies, Inc., detector: thermal conductivity detector).

The water content measurements from Karl Fischer titration were corrected using a calibration curve through the following procedures (1) to (6).
(1) The water content in an organic solvent that is the same as the organic solvent in each of the solutions to be measured (propylene glycol if the organic solvent in the solution was propylene glycol (PG), and hexylene glycol if the organic solvent in the solution was hexylene glycol (HG)) was measured by Karl Fischer titration. Next, a little amount of water was added to this organic solvent to prepare five solutions of different water contents (hereinafter may be referred to as “water/organic solvent solutions”). The amount of water added to the organic solvent was selected so that the water contents in the water/organic solvent solutions were five levels of 0.25 to 5.0 mass % (0.25 mass %, 0.50 mass %, 1.0 mass %, 2.0 mass % and 5.0 mass %). When the water contents in the five prepared water/organic solvent solutions were measured by Karl Fischer titration, it was confirmed that the obtained values well matched the theoretical values calculated from the water content in the organic solvent and the amount of the added water.
(2) Each of the five water/organic solvent solutions prepared in (1) is analyzed by gas chromatography (GC), so that GC charts including the peaks of water and the organic solvents were obtained. When the areas of the peaks of water in the obtained GC charts were plotted on the vertical axis (Y), and the water contents in the water/organic solvent solutions (theoretical values calculated from the water content in the organic solvent and the amount of the added water) were plotted on the horizontal axis (X), both had a good linear correlation. A regression line was calculated by the least squares using Y as a dependent variable and X as an explanatory variable, so that a calibration curve that gives the water content from the areas of the peaks of water in the GC charts (first calibration curve) was obtained.
(3) A little amount of a concentrated aqueous solution of QAH, which was the same as the quaternary ammonium hydroxide (QAH) in the solution to be measured (a 25 mass % TMAH aqueous solution if QAH in the solution was TMAH, a 20 mass % TEAH aqueous solution if QAH in the solution was TEAH, a 10 mass % TPAH aqueous solution if QAH in the solution was TPAH, and a 10 mass % TBAH aqueous solution if QAH in the solution was TBAH), was added to the organic solvent same as the organic solvent in the solution to be measured, so that five mixed solutions were prepared as standard solutions. The water content in the organic solvent was accurately measured by Karl Fischer titration in (1). The QAH concentration in the concentrated aqueous solution of QAH was accurately measured with an automatic potentiometric titration apparatus (this also determined the water content in the concentrated aqueous solution of QAH at the same time). The mixing mass ratio of the organic solvent and the concentrated aqueous solution of QAH was selected so that the water contents in the mixed solutions were five levels that were the same as in (1), that is, 0.25 to 5.0 mass %.
(4) The water contents in the standard solutions were determined by GC. That is, each of the five standard solutions prepared in (3) was analyzed by gas chromatography, so that the water content in each of the standard solutions was obtained from the areas of the peaks of water in the GC charts, using the first calibration curve obtained in (2). It was confirmed that these water content measurements by GC well matched theoretical values of the water contents in the standard solutions which were calculated from the water content in the organic solvent, the water content in the concentrated aqueous solution of QAH, and the mixing mass ratio of the organic solvent and the concentrated aqueous solution of QAH.
(5) The water content in each of the five standard solutions prepared in (3) was measured by Karl Fischer titration. The water contents of the standard solutions measured by Karl Fischer titration were plotted on the vertical axis (Y), and the water contents of the standard solutions, which were measured by GC in (3), were plotted on the horizontal axis (X). A regression line was calculated by the least squares using Y as a dependent variable and X as an explanatory variable, so that a calibration curve for correcting a water content measurement of the organic solvent solution containing QAH and water from Karl Fischer titration to that from GC (second calibration curve) was obtained.
(6) The water content of the actual solution to be measured was measured by Karl Fischer titration. The obtained measurement was corrected to the water content measured by GC, using the second calibration curve obtained in (5).

The metal impurity contents in the obtained solutions were measured by inductively coupled plasma mass spectrometry (ICP-MS) using ICP-MS 7500cx manufactured by Agilent Technologies, Inc. After the solutions were pretreated using a pretreatment cartridge for removing a cation, the amounts of chloride ions in the obtained solutions were measured by ion exchange chromatography using ion chromatography ICS-1100 manufactured by Thermo Fisher Scientific K.K. (column: Dionex (registered trademark) Ionpac (registered trademark) AS7 anion exchange column, eluent: additive-containing NaOH aqueous solution, detector: conductivity detector).

### (Thin Film Evaporation Apparatus)

As a thin film evaporation apparatus, a commercially available flowing-down-type short-path thin film evaporation apparatus (KD-10 manufactured by UIC GmbH, heating surface area: 0.1 m²) was used as purchased or modified. The configuration of the apparatus in each of the examples and comparative examples was as follows.

Apparatus C: as shown in FIG. 4 (thin film evaporation apparatus 10C), the apparatus C comprised, in the order mentioned from the upstream side, the raw material reservoir 31, the valve 32, the conduit 3, the raw material pump 4, the preheater 5, the degasser 6, the evaporation vessel (including the roller wiper 21 and the inside condenser 22) 37, the glass conduits for flow rate confirmation (on the residue and distillate sides) 8 and 9, the gear pumps (on the residue and distillate sides) 10 and 11, the residue recovery vessel 12, the distillate recovery vessel 13, as well as the vacuum pump (rotary pump and roots pump) 15 and the cold trap 14, and other conduits, valves etc., connecting the foregoing.

As for the materials of the liquid-contacting portions in the apparatus C, the roller wiper 21 was made of a composite material of PTFE and glass fiber, the liquid-contacting portions other than the roller wiper 21 were made of stainless steel (SUS304, SUS316L, SUS316Ti, SUS630 or any equivalent), and the residue recovery vessel 12 and the distillate recovery vessel 13 were made of PE. The area of the heating surface 24 was 0.1 m².

Apparatus A: as shown in FIG. 1 (thin film evaporation apparatus 10A), the apparatus A had the structure such that the raw material gear pump 4, the preheater 5 and the degasser 6 were removed from the structure of the apparatus C, and the valve 32 was changed to a needle valve.

As for the materials of the liquid-contacting portions in the apparatus A, the raw material reservoir 31 was made of PE, the conduit 33 was made of PFA, and the needle valve 32 for adjusting the flow rate was made of PTFE. The material of the roller wiper 21 inside the evaporation vessel 37 was changed from the composite material of PTFE and glass fiber to PEEK (containing no glass fiber).

A sample of a small piece was cut out of the resin of each of PE, PFA, PTFE and PEEK used in the liquid-contacting portions of the apparatus A, and decomposed, and metal impurities of Na, Ca, Al, and Fe in each resin were measured by ICP-MS. As a result, each of Na, Ca, Al, and Fe was in an amount of no more than 1 mass ppm.

Apparatus B: as shown in FIG. 3 (thin film evaporation apparatus 10B), the apparatus B was such that the glass conduit for flow rate confirmation (on the residue side) 8 was removed from the apparatus A, and conduits 38 from the outlet of the evaporation vessel 37 to the residue recovery vessel 12 and to the distillate recovery vessel 13 were made of PFA.

In all the apparatuses A to C, the degrees of vacuum in the systems were each measured by a vacuum gauge (not shown) disposed between the cold trap 14 and the vacuum pump 15.

The abbreviations and sources of the materials used in each of the examples and comparative examples are as follows:
25 mass % TMAH aqueous solution: TMAH aqueous solution where the concentration of a tetramethylammonium hydroxide (TMAH) was 25 mass % (manufactured by TOKUYAMA CORPORATION)
PG: propylene glycol (manufactured by AGC Inc.)
HG: hexylene glycol (manufactured by Mitsui Chemicals, Inc.)

A TEAH aqueous solution, a TPAH aqueous solution, and a TBAH aqueous solution (all manufactured by Wako Pure Chemical Industries, Ltd.) were each purified by a dual-chamber electrolysis method of an aqueous solution system, and prepared and used a TEAH aqueous solution where the TEAH concentration was 20 mass % (20 mass % TEAH aqueous solution), a TPAH aqueous solution where the TPAH concentration was 10 mass % (10 mass % TPAH aqueous solution), and a TBAH aqueous solution where the TBAH concentration was 10 mass % (10 mass % TBAH aqueous solution) as aqueous quaternary ammonium hydroxide solutions as the raw materials. The aqueous quaternary ammonium hydroxide solutions and the water-soluble organic solvents of the raw materials were stored in a room at 23° C. in temperature, and thereafter used for preparing raw material mixture liquids.

The metal impurity content in the raw material mixture liquids each used in the examples and comparative examples are shown in Table 1. In Table 1, “<1” means that the content took a value less than 1 mass ppb.

TABLE 1

| Raw material | Metal impurity content (mass ppb) | | | | | | | | | | |
|---|---|---|---|---|---|---|---|---|---|---|---|
| mixture liquid | Na | Mg | Al | K | Ca | Ti | Cr | Mn | Fe | Ni | Zn |
| comparative | <1 | <1 | 3 | 1 | 1 | <1 | <1 | <1 | 3 | <1 | <1 |
| example 1 | | | | | | | | | | | |
| exemple 1 | <1 | <1 | 3 | 1 | 1 | <1 | <1 | <1 | 3 | <1 | <1 |
| example 2 | <1 | <1 | 3 | 1 | 1 | <1 | <1 | <1 | 3 | <1 | <1 |
| example 3 | <1 | <1 | 3 | 1 | 1 | <1 | <1 | <1 | 3 | <1 | <1 |
| example 4 | <1 | <1 | 3 | 1 | 1 | <1 | <1 | <1 | 3 | <1 | <1 |
| example 5 | <1 | <1 | 3 | 1 | 1 | <1 | <1 | <1 | 3 | <1 | <1 |
| example 6 | <1 | <1 | 3 | 1 | 1 | <1 | <1 | <1 | 3 | <1 | <1 |
| example 7 | <1 | <1 | 3 | 1 | 1 | <1 | <1 | <1 | 3 | <1 | <1 |
| exarnple 8 | 4 | <1 | 5 | 6 | 2 | <1 | <1 | <1 | 4 | <1 | <1 |
| example 9 | 5 | <1 | 5 | 7 | 3 | <1 | <1 | <1 | 4 | <1 | <1 |
| example 10 | 8 | <1 | 6 | 8 | 3 | <1 | <1 | <1 | 4 | <1 | <1 |

### Comparative Example 1

Thin film evaporation was performed using the apparatus C (thin film evaporation apparatus 10C (FIG. 4)), to produce an organic solvent solution of a quaternary ammonium hydroxide.

The conduits of the apparatus were disassembled, washed, and assembled in advance. Thereafter a TMAH aqueous solution where the TMAH concentration was 25 mass %, and ultrapure water were each alternately circulated around the conduits twice, to wash the conduits.

A raw material mixture liquid prepared by mixing 4 kg of the 25 mass % TMAH aqueous solution and 20 kg of PG in a clean bottle made of PE was put in the raw material reservoir made of SUS304 (mixing mass ratio of TMAH aqueous solution/PG=1/5). Thin film evaporation was performed under the conditions of: preheater temperature 70° C.; temperature of the raw material mixture liquid right before the raw material mixture liquid entered the evaporation vessel 68° C.; temperature of the heating surface of the evaporation vessel (heat medium temperature) 100° C.; degree of vacuum 1900 Pa; and feed rate 7.0 kg/hour (feed rate per unit area of the heating surface: 70 kg/hour·m²), so that a PG solution containing TMAH (approximately 8 kg) was obtained in the residue recovery vessel. Each of the conditions is shown in Table 2. In Table 2, as for the raw material mixture liquid, “mixing ratio” means the mixing mass ratio of the aqueous quaternary ammonium hydroxide solution and the water-soluble organic solvent (aqueous quaternary ammonium hydroxide solution/water-soluble organic solvent). The TMAH concentration, the water content, the metal impurity content, and the amount of chloride ions in each of the obtained solutions are shown in Table 3. In Table 3, “TXAH concentration” means the concentration of a quaternary ammonium hydroxide, and “<1” means that the content took a value less than 1 mass ppb.

### Example 1

Thin film evaporation (step (a)) was performed using the apparatus A (thin film evaporation apparatus 10A (FIG. 1)), to produce an organic solvent solution of a quaternary ammonium hydroxide.

The conduits of the apparatus were disassembled, washed, and assembled in advance, Thereafter a TMAH aqueous solution where the TMAH concentration was 25 mass %, and ultrapure water were each alternately circulated around the conduits twice, to wash the conduits (step (b)).

A raw material mixture liquid prepared by mixing 4 kg of the 25 mass % TMAH aqueous solution and 16 kg of PG in a clean bottle made of PE was put in the raw material reservoir made of PE (mixing mass ratio of TMAH aqueous solution/PG=1/4). Thin film evaporation was performed under the conditions of: temperature of the raw material mixture liquid right before the raw material mixture liquid entered the evaporation vessel 23° C.; temperature of the heating surface of the evaporation vessel (heat medium temperature) 100° C.; degree of vacuum 600 Pa; and feed rate 10.0 kg/hour (feed rate per unit area of the heating surface: 100 kg/hour·m²), so that a PG solution containing TMAH (approximately 5 kg) was obtained in the residue recovery vessel (step (a)). The conditions and the results are shown in Tables 2 and 3.

### Example 2

The apparatus A was cleaned in the same manner as in Example 1 (step (b)), and thereafter thin film evaporation (step (a)) was performed, using the apparatus A (thin film evaporation apparatus 10A (FIG. 1)), to produce an organic solvent solution of a quaternary ammonium hydroxide.

A raw material mixture liquid prepared by mixing 4 kg of the 25 mass % TMAH aqueous solution and 16 kg of PG in a clean bottle made of PE was put in the raw material reservoir made of PE (mixing mass ratio of TMAH aqueous solution/PG=1/4). Thin film evaporation was performed under the conditions of: temperature of the raw material mixture liquid right before the raw material mixture liquid entered the evaporation vessel 23° C.; temperature of the heating surface of the evaporation vessel (heat medium temperature) 105° C.; degree of vacuum 500 Pa; and feed rate 7.0 kg/hour (feed rate per unit area of the heating surface: 70 kg/hour·m²), so that a PG solution containing TMAH (approximately 4 kg) was obtained in the residue recovery vessel. The conditions and the results are shown in Tables 2 and 3.

### Example 3

The apparatus B was cleaned in the same manner as in Example 1 (step (b)), and thereafter thin film evaporation (step (a)) was performed, using the apparatus B (thin film evaporation apparatus 10B (FIG. 3)), to produce an organic solvent solution of a quaternary ammonium hydroxide.

A raw material mixture liquid prepared by mixing 4 kg of the 25 mass % TMAH aqueous solution and 16 kg of PG in a clean bottle made of PE was put in the raw material reservoir made of PE (mixing mass ratio of TMAH aqueous solution/PG=1/4). Thin film evaporation was performed under the conditions of: temperature of the raw material mixture liquid right before the raw material mixture liquid entered the evaporation vessel 23° C.; temperature of the heating surface of the evaporation vessel (heat medium temperature) 105° C.; degree of vacuum 500 Pa; and feed rate 5.0 kg/hour (feed rate per unit area of the heating surface: 50 kg/hour·m²), so that a PG solution containing TMAH (approximately 4 kg) was obtained in the residue recovery vessel. The conditions and the results are shown in Tables 2 and 3.

### Example 4

Thin film evaporation was performed in the same manner as in Example 3 except that the degree of vacuum was 300 Pa, so that a PG solution containing TMAH (approximately 3 kg) was obtained in the residue recovery vessel. The conditions and the results are shown in Tables 2 and 3.

### Example 5

Thin film evaporation was performed in the same manner as in Example 3 except that the temperature of the heating surface (heat medium temperature) was 80° C., the degree of vacuum was 16 Pa, and the feed rate was 2.5 kg/hour (the feed rate per unit area of the heating surface was 25 kg/hour·m²), so that a PG solution containing TMAH (approximately 4 kg) was obtained in the residue recovery vessel. The conditions and the results are shown in Tables 2 and 3.

### Example 6

The apparatus B was cleaned in the same manner as in Example 1 (step (b)), and thereafter thin film evaporation (step (a)) was performed, using the apparatus B (thin film evaporation apparatus 10B (FIG. 3)), to produce an organic solvent solution of a quaternary ammonium hydroxide.

A raw material mixture liquid prepared by mixing 4 kg of the 25 mass % TMAH aqueous solution and 8 kg of PG in a clean bottle made of PE was put in the raw material reservoir made of PE (mixing mass ratio of aqueous solution/PG=1/2). Thin film evaporation was performed under the conditions of: temperature of the raw material mixture liquid right before the raw material mixture liquid entered the evaporation vessel 23° C.; temperature of the heating surface of the evaporation vessel (heat medium temperature) 105° C.; degree of vacuum 16 Pa; and feed rate 2.5 kg/hour (feed rate per unit area of the heating surface: 25 kg/hour m²), so that a PG solution containing TMAH (approximately 3 kg) was obtained in the residue recovery vessel. The conditions and the results are shown in Tables 2 and 3.

### Example 7

The apparatus B was cleaned in the same manner as in Example 1 (step (b)), and thereafter thin film evaporation (step (a)) was performed, using the apparatus B (thin film evaporation apparatus 10B (FIG. 3)), to produce an organic solvent solution of a quaternary ammonium hydroxide.

A raw material mixture liquid prepared by mixing 4 kg of the 25 mass % TMAH aqueous solution and 16 kg of HG in a clean bottle made of PE was put in the raw material reservoir made of PE (mixing mass ratio of TMAH aqueous solution/HG=1/4). Thin film evaporation was performed under the conditions of: temperature of the raw material mixture liquid right before the raw material mixture liquid entered the evaporation vessel 23° C.; temperature of the heating surface of the evaporation vessel (heat medium temperature) 105° C.; degree of vacuum 500 Pa; and feed rate 7.0 kg/hour (feed rate per unit area of the heating surface: 70 kg/hour·m²), so that a HG solution containing TMAH (approximately 4 kg) was obtained in the residue recovery vessel. The conditions and the results are shown in Tables 2 and 3.

### Example 8

The apparatus B (thin film evaporation apparatus 10B (FIG. 3)) was cleaned through the same procedures as in Example 1 (step (b)). Instead of the TMAH aqueous solution, the 20 mass % TEAH aqueous solution was used as a cleaning solution. Thereafter thin film evaporation (step (a)) was performed through the following procedures, to produce an organic solvent solution of a quaternary ammonium hydroxide.

A raw material mixture liquid prepared by mixing 4 kg of the 20 mass % TEAH aqueous solution and 16 kg of PG in a clean bottle made of PE was put in the raw material reservoir made of PE (mixing mass ratio of TEAH aqueous solution/PG=1/4). Thin film evaporation was performed under the conditions of: temperature of the raw material mixture liquid right before the raw material mixture liquid entered the evaporation vessel 23° C.; temperature of the heating surface of the evaporation vessel (heat medium temperature) 105° C.; degree of vacuum 100 Pa; and feed rate 5.0 kg/hour (feed rate per unit area of the heating surface: 50 kg/hour·m²), so that a PG solution containing TEAH (approximately 4 kg) was obtained in the residue recovery vessel. The conditions and the results are shown in Tables 2 and 3.

### Examples 9 and 10

Thin film evaporation was performed in the same manner as in Example 8 except that the TEAH aqueous solution used for washing, and preparing the raw material mixture liquid was changed to the 10 mass % TPAH aqueous solution (Example 9), or the 10 mass % TBAH aqueous solution (Example 10), so that a PG solution containing TPAH (approximately 4 kg) or a PG solution containing TBAH (approximately 4 kg) was obtained in the residue recovery vessel. The conditions and the results are shown in Tables 2 and 3.

TABLE 2

| | Raw material mixture liquid | | | | | | | | | |
|---|---|---|---|---|---|---|---|---|---|---|
| | | | | | amount | | | | | |
| | | | | | of water- | | | | | |
| | | | | | soluble | | | | | |
| | | | | water | organic | Conditions for thin film evaporation | | | | |
| | | | | content | solvent | | temperature | temperature | | |
| | | | | in raw | in raw | | of raw | of heating | | |
| | | water- | | material | material | | material | surface | degree | |
| | quaternary | soluble | | mixture | mixture | | (first | (second | of | |
| | ammonium | organic | mixing | liquid | liquid | used | temperature) | temperature) | vacuum | feed rate |
| | hydroxide | solvent | ratio | mass % | mass % | apparatus | ° C. | ° C. | Pa | kg/hour |
| comparative | TMAH | PG | 1/5 | 12.5 | 83.3 | C | 68 | 100 | 1900 | 7.0 |
| example 1 | | | | | | | | | | |
| example 1 | TMAH | PG | 1/4 | 15.0 | 80.0 | A | 23 | 100 | 600 | 10.0 |
| example 2 | TMAH | PG | 1/4 | 15.0 | 80.0 | A | 23 | 105 | 500 | 7.0 |
| example 3 | TMAH | PG | 1/4 | 15.0 | 80.0 | B | 23 | 105 | 500 | 5.0 |
| example 4 | TMAH | PG | 1/4 | 15.0 | 80.0 | B | 23 | 105 | 300 | 5.0 |
| example 5 | TMAH | PG | 1/4 | 15.0 | 80.0 | B | 23 | 80 | 16 | 2.5 |
| example 6 | TMAH | PG | 1/2 | 25.0 | 66.7 | B | 23 | 105 | 16 | 2.5 |
| example 7 | TMAH | HG | 1/4 | 15.0 | 80.0 | B | 23 | 105 | 500 | 7.0 |
| example 8 | TEAH | PG | 1/4 | 16.0 | 80.0 | B | 23 | 105 | 100 | 5.0 |
| example 9 | TPAH | PG | 1/4 | 18.0 | 80.0 | B | 23 | 105 | 100 | 5.0 |
| example 10 | TBAH | PG | 1/4 | 18.0 | 0.0 | B | 23 | 105 | 100 | 5.0 |

TABLE 3

| | TXAH | Water | Impurity content | | | | | | | | | | | | |
|---|---|---|---|---|---|---|---|---|---|---|---|---|---|---|---|
| | concentration | content | Na | Mg | Al | K | Ca | Ti | Cr | Mn | Fe | Ni | Cu | Zn | Cl |
| | mass % | mass % | mass ppb | | | | | | | | | | | | |
| comparative | 12.5 | 2.0 | 269 | 7 | 21 | 22 | 103 | 3 | 10 | 6 | 150 | 8 | 2 | 31 | 334 |
| example 1 | | | | | | | | | | | | | | | |
| example 1 | 18.5 | 0.98 | 49 | 3 | 10 | 7 | 31 | 1 | 6 | 2 | 38 | 4 | 1 | 10 | 63 |
| example 2 | 24.1 | 0.56 | 14 | <1 | 8 | 4 | 15 | <1 | 3 | <1 | 13 | 2 | <1 | 4 | 59 |
| example 3 | 24.5 | 0.52 | 8 | <1 | 7 | 3 | 13 | <1 | 2 | <1 | 9 | 1 | <1 | 3 | 35 |
| example 4 | 27.8 | 0.50 | 8 | <1 | 7 | 3 | 12 | <1 | 2 | <1 | 9 | 1 | <1 | 3 | 32 |
| example 5 | 25.7 | 0.29 | 7 | <1 | 7 | 3 | 11 | <1 | 1 | <1 | 8 | <1 | <1 | 3 | 28 |
| example 6 | 32.1 | 0.30 | 6 | <1 | 6 | 2 | 11 | <1 | 2 | <1 | 11 | 2 | <1 | 3 | 30 |
| example 7 | 24.7 | 0.58 | 10 | <1 | 8 | 4 | 13 | <1 | 5 | <1 | 20 | 3 | <1 | 3 | 50 |
| example 8 | 19.8 | 0.84 | 69 | 1 | 20 | 72 | 33 | <1 | 3 | <1 | 18 | 2 | <1 | 13 | 65 |
| example 9 | 9.6 | 0.94 | 74 | 1 | 23 | 81 | 39 | <1 | 4 | <1 | 21 | 3 | <1 | 12 | 58 |
| example 10 | 9.0 | 0.98 | 90 | 1 | 30 | 98 | 42 | <1 | 4 | <1 | 20 | 3 | <1 | 8 | 10 |

The contents of Na, Ca and Fe that were metal impurities in the TMAH-containing PG solution obtained in comparative example 1 were each more than 100 mass ppb, and the chlorine impurity content therein was also more than 100 mass ppb.

In examples 1 to 10, the organic solvent solutions of a quaternary ammonium hydroxide of a high degree of purity were obtained: the quaternary ammonium hydroxide in each of the organic solvent solutions had a water content of no more than 1.0 mass %, a metal impurity content of no more than 100 ppb in terms of each metal, and a chlorine impurity content of no more than 100 ppb. Such an organic solvent solution of a quaternary ammonium hydroxide of a high degree of purity was not obtained conventionally. The water content could be no more than 0.3 mass %, the metal impurity content could be no more than 20 mass ppb in terms of each metal, and the chlorine impurity content could be no more than 50 mass ppb, according to conditions of thin film evaporation (Examples 5 and 6). The organic solvent solutions of a quaternary ammonium hydroxide obtained in examples 1 to 10 each had a concentration and purity so as to be able to be used as they were as treatment liquid compositions for semiconductor production. It is also possible to obtain treatment liquid compositions for semiconductor production by further carrying out the step (iii) in the composition production method according to the third aspect of the present invention (see the section 3.3) on the organic solvent solutions of a quaternary ammonium hydroxide obtained in examples 1 to 10.

### REFERENCES SIGNS LIST

3, 33 raw material conduit
4 raw material gear pump
5 preheater
6 degasser
8, 9 glass conduit for flow rate confirmation
10 feed pump (gear pump (on the residue side))
11 feed pump (gear pump (on the distillate side))
12 residue recovery vessel
13 distillate recovery vessel
14 cold trap
15 vacuum pump
21 wiper (roller wiper)
22 condenser (inside condenser)
23 raw material mixture liquid
24 heating surface
25 (circulating) heat medium
26 (circulating) refrigerant
31 raw material reservoir
32 valve (needle valve)
37 evaporation vessel
38 conduit

1. A method for producing an organic solvent solution of a quaternary ammonium hydroxide,
the solution having a water content of no more than 1.0 mass % on the basis of the total mass of the solution,
contents of Na, Mg, Al, K, Ca, Ti, Cr, Mn, Fe, Ni, Cu, and Zn in the solution each being no more than 100 mass ppb on the basis of the total mass of the solution,
the solution having a Cl content of no more than 100 mass ppb on the basis of the total mass of the solution,
the method comprising:
(a) subjecting a raw material mixture liquid to a thin film evaporation by means of a thin film evaporation apparatus, to remove water from the raw material mixture liquid,
the raw material mixture liquid comprising:
at least one quaternary ammonium hydroxide;
water; and
at least one first organic solvent dissolving the quaternary ammonium hydroxide, the at least one first organic solvent being at least one water-soluble organic solvent each having a plurality of hydroxy groups,
the thin film evaporation apparatus comprising:
an evaporation vessel;
a raw material reservoir storing the raw material mixture liquid; and
a raw material conduit transferring the raw material mixture liquid from the raw material reservoir to the evaporation vessel,
wherein liquid-contacting portions of inner surfaces of the raw material reservoir and the raw material conduit are each made of resin.

2. The method according to claim 1,
wherein contents of Na, Ca, Al, and Fe in the resin constituting the liquid-contacting portions are each no more than 1 mass ppm.

3. The method according to claim 1, the method further comprising:
(b) prior to the (a), washing the liquid-contacting portions with a solution comprising the at least one quaternary ammonium hydroxide.

4. The method according to claim 1,
wherein each of the at least one first organic solvent has a boiling point of 150 to 300° C.

5. The method according to claim 1,
wherein the at least one first organic solvent is at least one alcohol selected from divalent alcohols and trivalent alcohols, wherein each of the divalent alcohols and trivalent alcohols consists of carbon atoms, hydrogen atoms, and oxygen atoms, and wherein each of the divalent alcohols and trivalent alcohols has a boiling point of 150 to 300° C.

6. The method according to claim 1,
wherein the at least one first organic solvent is ethylene glycol, propylene glycol, diethylene glycol, dipropylene glycol, tripropylene glycol, hexylene glycol, or glycerin, or any combination thereof.

7. The method according to claim 1,
the raw material mixture liquid comprising, on the basis of the total mass of the mixture liquid:
40 to 85 mass % of the at least one first organic solvent;
2.0 to 30 mass % of the at least one quaternary ammonium hydroxide; and
10 to 30 mass % of the water.

8. The method according to claim 1,
wherein contents of Na, Mg, Al, K, Ca, Ti, Cr, Mn, Fe, Ni, Cu, and Zn in the raw material mixture liquid are each no more than 50 mass ppb on the basis of the total mass of the raw material mixture liquid; and
a content of Cl in the raw material mixture liquid is no more than 50 mass ppb on the basis of the total mass of the raw material mixture liquid.

9. The method according to claim 1,
the thin film evaporation apparatus being a flowing-down-type thin film evaporation apparatus,
the evaporation vessel comprising an inner wall surface,
the thin film evaporation apparatus further comprising:
a first flow path introducing the raw material mixture liquid into the evaporation vessel from an upper part of the evaporation vessel,
the raw material mixture liquid introduced from the first flow path into the evaporation vessel flowing down as a liquid film along the inner wall surface of the evaporation vessel,
the thin film evaporation apparatus further comprising:
a heating surface arranged in the inner wall surface, the heating surface heating the liquid film flowing down along the inner wall surface;
a condenser arranged inside evaporation vessel, the condenser cooling and liquefying a vapor from the liquid film;
a second flow path recovering a distillate liquefied by the condenser from the evaporation vessel; and
a third flow path recovering a residue from the evaporation vessel, the residue not evaporating but flowing down from the heating surface,
wherein the thin film evaporation is carried out under conditions such that:
the raw material mixture liquid has a first temperature right before entering into the evaporation vessel, wherein the first temperature is no more than 70° C.;
the heating surface has a second temperature of 60 to 140° C., wherein the second temperature is higher than the first temperature; and
a degree of vacuum in the evaporation vessel is no more than 600 Pa.

10. The method according to claim 9,
the thin film evaporation apparatus further comprising:
a wiper being arranged in the evaporation vessel and rotating along the inner wall surface,
wherein the raw material mixture liquid introduced into the evaporation vessel from the first flow path is spread on the inner wall surface with the wiper, to form the liquid film.

11. The method according to claim 9,
wherein the heating surface is made of a metal, wherein the metal is exposed at the heating surface.

12. A method for producing a treatment liquid composition for semiconductor production, the composition comprising:
at least one quaternary ammonium hydroxide; and
at least one first organic solvent dissolving the at least one quaternary ammonium hydroxide, the at least one first organic solvent being at least one water-soluble organic solvent each having a plurality of hydroxy groups,
wherein a water content in the composition is no more than 1.0 mass % on the basis of the total mass of the composition;
contents of Na, Mg, Al, K, Ca, Ti, Cr, Mn, Fe, Ni, Cu, and Zn in the composition are each no more than 100 mass ppb on the basis of the total mass of the composition; and
a content of Cl in the composition is no more than 100 mass ppb on the basis of the total mass of the composition,
the method comprising:
(i) obtaining an organic solvent solution of the at least one quaternary ammonium hydroxide by a method as in claim 1,
(ii) knowing a total concentration of the at least one quaternary ammonium hydroxide in the organic solvent solution; and
(iii) adding at least one second organic solvent to the organic solvent solution, to adjust the total concentration of the at least one quaternary ammonium hydroxide in the organic solvent solution, wherein each of the at least one second organic solvent has a water content of no more than 1.0 mass % on the basis of the total mass of the second organic solvent, and wherein contents of Na, Mg, Al, K, Ca, Ti, Cr, Mn, Fe, Ni, Cu, and Zn in each of the at least one second organic solvent are each no more than 100 mass ppb on the basis of the total mass of the second organic solvent, and wherein each of the at least one second organic solvent has a Cl content of no more than 100 mass ppb on the basis of the total mass of the second organic solvent.

13. A treatment liquid composition for semiconductor production, the composition comprising:
at least one quaternary ammonium hydroxide; and
at least one first organic solvent dissolving the at least one quaternary ammonium hydroxide, the at least one first organic solvent being at least one water-soluble organic solvent each having a plurality of hydroxy groups,
wherein a water content in the composition is no more than 1.0 mass % on the basis of the total mass of the composition;
contents of Na, Mg, Al, K, Ca, Ti, Cr, Mn, Fe, Ni, Cu, and Zn in the composition are each no more than 100 mass ppb on the basis of the total mass of the composition;
a content of Cl in the composition is no more than 100 mass ppb on the basis of the total mass of the composition; and
a ratio of the water content (unit: mass %) in the composition to a total content of the at least one quaternary ammonium hydroxide (unit: mass %) in the composition (water/quaternary ammonium hydroxide) is no more than 0.42,
wherein the composition is produced by a process, the process comprising:
(i) obtaining an organic solvent solution of the at least one quaternary ammonium hydroxide by a method as in claim 1.

14. The composition according to claim 13,
the process further comprising:
(ii) knowing a total concentration of the at least one quaternary ammonium hydroxide in the organic solvent solution; and
(iii) adding at least one second organic solvent to the organic solvent solution, to adjust the total concentration of the at least one quaternary ammonium hydroxide in the organic solvent solution, wherein each of the at least one second organic solvent has a water content of no more than 1.0 mass % on the basis of the total mass of the second organic solvent, and wherein contents of Na, Mg, Al, K, Ca, Ti, Cr, Mn, Fe, Ni, Cu, and Zn in each of the at least one second organic solvent are each no more than 100 mass ppb on the basis of the total mass of the second organic solvent, and wherein each of the at least one second organic solvent has a Cl content of no more than 100 mass ppb on the basis of the total mass of the second organic solvent.

15. The composition according to claim 13,
wherein the (i) is obtaining the organic solvent solution of the at least one quaternary ammonium hydroxide by a method as in claim 7.

16. The composition according to claim 13,
wherein the (i) is obtaining the organic solvent solution of the at least one quaternary ammonium hydroxide by a method as in claim 8.

17. The composition according to claim 13,
wherein the (i) is obtaining the organic solvent solution of the at least one quaternary ammonium hydroxide by a method as in claim 9.

18. The composition according to claim 17,
wherein the (i) is obtaining the organic solvent solution of the at least one quaternary ammonium hydroxide by a method as in claim 10.

19. The composition according to claim 17,
wherein the (i) is obtaining the organic solvent solution of the at least one quaternary ammonium hydroxide by a method as in claim 11.

20. The composition according to claim 13,
wherein the water content in the composition is no more than 0.5 mass % on the basis of the total mass of the composition;
the contents of Na, Mg, Al, K, Ca, Ti, Cr, Mn, Fe, Ni, Cu, and Zn in the composition are each no more than 50 mass ppb on the basis of the total mass of the composition; and
the content of Cl in the composition is no more than 80 mass ppb on the basis of the total mass of the composition.

21. The composition according to claim 13,
wherein the water content in the composition is no more than 0.3 mass % on the basis of the total mass of the composition;
the contents of Na, Mg, Al, K, Ca, Ti, Cr, Mn, Fe, Ni, Cu, and Zn in the composition are each no more than 20 mass ppb on the basis of the total mass of the composition; and
the content of Cl in the composition is no more than 50 mass ppb on the basis of the total mass of the composition.

22. The composition according to claim 13,
wherein the total content of the at least one quaternary ammonium hydroxide in the composition is no less than 5.0 mass % on the basis of the total mass of the composition.

23. The composition according to claim 13,
wherein a ratio of the Na content (unit: mass ppb) in the composition to the total content of the at least one quaternary ammonium hydroxide (unit: mass %) in the composition (Na/quaternary ammonium hydroxide) is 0.0001 to 42; and
a ratio of the Ca content (unit: mass ppb) in the composition to the total content of the at least one quaternary ammonium hydroxide (unit: mass %) in the composition (Ca/quaternary ammonium hydroxide) is 0.0001 to 42.

24. The composition according to claim 13,
wherein a ratio of the Cl content (unit: mass ppb) in the composition to the total content of the at least one quaternary ammonium hydroxide (unit: mass %) in the composition (Cl/quaternary ammonium hydroxide) is 0.0001 to 42.
